(19) United States
(12) Patent Application Publication
(10) Pub. No.: US 2008/0108513 A1
(43) Pub. Date: May 8, 2008

(54) Cellular Arrays

(75) Inventors: Angela K. Pannier, Lincoln, NE (US); Eric A. Ariazi, Blue Bell, PA (US); V. Craig Jordan, Rydal, PA (US); Lonnie D. Shea, Chicago, IL (US)

Correspondence Address: Casimir Jones, S.C., 440 Science Drive, Suite 203, Madison, WI, 53711, US

(73) Assignee: Northwestern University, Evanston, IL (US)

(21) Appl. No.: 11/809,772

(22) Filed: Jun. 1, 2007

Related U.S. Application Data
(60) Provisional application No. 60/810,102, filed on Jun. 1, 2006.
(60) Provisional application No. 60/810,110, filed on Jun. 1, 2006.

(51) Int. Cl. C40B 40/08; C12N 15/87; C40B 50/06

(52) U.S. Cl. 506/17; 435/455; 506/26

(57) ABSTRACT

The present invention relates to characterizing transcription within cells. In particular, the present invention provides transfected cell arrays (e.g., two-dimensional and/or three-dimensional arrays) and systems, kits and methods utilizing the same (e.g., for transcriptional activity characterization). Compositions and methods of the present invention find use in, among other things, research, drug discovery and clinical (e.g., diagnostic, preventative and therapeutic) applications.

The present invention claims priority to U.S. Provisional Patent Application Ser. Nos. 60/810,102 and 60/810,110, filed Jun. 1, 2006, each of which is hereby incorporated by reference in its entirety.

This invention was made with government support under contracts W81XWH-05-1-0381 awarded by the United States Army Medical Research and Material Command (USAMRMC); RO1 GM066830 and CA89018-03 awarded by the National Institutes of Health; and BES0092701 awarded by the National Science Foundation. The government has certain rights in the invention.

### FIELD OF THE INVENTION

The present invention relates to characterizing transcription within cells. In particular, the present invention provides transfected cell arrays (e.g., two-dimensional and/or three-dimensional arrays) and systems, kits and methods utilizing the same (e.g., for transcriptional activity characterization). Compositions and methods of the present invention find use in, among other things, research, drug discovery and clinical (e.g., diagnostic, preventative and therapeutic) applications.

### BACKGROUND OF THE INVENTION

The human genome project has lead to an incredible wealth of information regarding nucleic acid sequence. However, an emerging challenge is to identify gene products and to determine the functional roles of the genes and gene products.

### SUMMARY OF THE INVENTION

The present invention relates to characterizing transcription within cells. In particular, the present invention provides transfected cell arrays (e.g., two-dimensional and/or three-dimensional arrays) and systems, kits and methods utilizing the same (e.g., for transcriptional activity characterization). Compositions and methods of the present invention find use in, among other things, research, drug discovery and clinical (e.g., diagnostic, preventative and therapeutic) applications.

Accordingly, in some embodiments, the present invention provides a composition comprising a three-dimensional transfection cell array comprising an array of spatially controllable immobilized gels, wherein the gels comprise a crosslinkable biopolymer solution comprising cells exposed to transfection molecules. In some embodiments, the gels are formed by adding a solution comprising transfection molecules and cells to a crosslinkable biopolymer (e.g., alginate) solution, allowing beads to form, and placing the same within cell growth media. In some embodiments, the crosslinkable biopolymer solution comprises alginate and collagen. the present invention is not limited to any particular crosslinkable biopolymer solution. Indeed, a variety of crosslinkable biopolymer solutions are contemplated to be useful in the compositions and methods of the present invention including, but not limited to, acrylated poly(ethylene glycol) (PEG-Acryl)hydrogels and other biopolymer solutions described herein. In some embodiments, the composition further comprises a charge neutralizing reagent (e.g., polyethyleimine (PEI)). In some embodiments, the alginate is present at a final concentration of about 1.5%, although higher and lower percentages may be used (e.g., as described herein). In some embodiments, the collagen is present at a final concentration of about 0.2%, although higher and lower percentages may be used (e.g., as described herein) In some embodiments, PEI is present in an amount such that the nitrogen to phosphate ratio is 25. In some embodiments, the transfection molecules are nucleic acid sequences. In some embodiments, the nucleic acid sequences are located within expression vectors. In some embodiments, the transfection molecules comprise a normalization plasmid and a functional plasmid. In some embodiments, the normalization plasmid comprises a promoter region and a reporter molecule that allows cellular transfection efficiency to be normalized over the array. In some embodiments, the functional plasmid comprises a reporter molecule that is different from the reporter molecule of the normalization plasmid. The present invention is not limited by the type of reporter molecules utilized. Indeed, a variety of reporter molecules are described herein including, but not limited to, various luciferase reporters and fluorescent reporters (e.g., green fluorescent protein reporters).

In some embodiments, the present invention also provides, a method of transfecting cells comprising providing: a composition comprising a biopolymer comprising alginate and collagen, transfection molecules, and cells; and mixing the composition comprising biopolymer, transfection molecules and cells, in the presence of PEI, under conditions such that beads form; and placing the beads in cell growth media.

Embodiments of the present invention also provide a method of generating an array, comprising: forming holes in a first material to generate an array mold, attaching the array mold to a solid substrate to form a complex comprising a plurality of wells; depositing first nucleic acid vectors into the plurality of wells under conditions such that the first nucleic acid vectors are generally immobilized on the solid substrate, wherein the first nucleic acid vectors comprise nucleic acid sequences, and removing the array mold from the solid substrate to generate an array on the solid substrate. In some embodiments, the nucleic acid sequences comprise a reporter gene; and/or a promoter region configured to bind transcription factors. The present invention also provides arrays (e.g., two-dimensional transfection cell arrays) generated according to such a method. In some embodiments, the first material comprises PDMS. In some embodiments, the solid substrate comprises polystyrene. In some embodiments, method further comprises depositing second nucleic acid vectors into the plurality of wells, wherein the second nucleic acid vectors comprise nucleic acid comprising: 1) a second reporter gene; and 2) a promoter region that allows transfection efficiency to be normalized (e.g., relative to transcriptional activity) over the array. In some embodiments, the first nucleic acid vectors are immobilized on the solid substrate such that they can deliver the nucleic acid to a cell.

### DESCRIPTION OF THE DRAWINGS

FIG. 1 shows a multiwell dish format reporter gene assay to compare surface delivery to traditional bolus delivery. Surface delivery (B) of ERE reporter plasmid system (pERE(3×)TK-ffLUC and normalization plasmid pTK-rLUC) resulted in E₂-stimulated transcriptional responses in MCF-7 breast cancer cells similar to bolus delivery (A), reported as a ratio of firefly to renilla luciferase, with E₂ statistically inducing firefly luciferase expression 6-7 fold over vehicle control or the addition of FUL. Columns labeled with same letter designate conditions not statistically different; all other comparisons, p<0.001.

FIG. 2 shows the effect of DNA amount on E₂ activation of ERE reporter plasmid system (pERE(3×)TK-ffLUC and normalization plasmid pTK-rLUC) delivered to MCF-7 breast cancer cells. Total amount of DNA added to the surface (B) or delivered as a bolus (A), in the presence of 10⁻⁹ M E₂, resulted in a similar dose-response effect. Columns labeled with same letter designate conditions not statistically different; all other comparisons, p<0.01 for (A), p<0.05 for (B).

FIG. 3 shows the effect of complexing agent and E₂ dose response on the ERE reporter plasmid system (pERE(3×)TK-ffLUC and normalization plasmid pTK-rLUC). Bolus and surface delivery of Lipofectamine 2000 complexes (A) resulted in induction profiles that were not statistically different from each other, for each concentration of E₂. Bolus delivery of Effectene complexes (B) resulted in statistically higher induction (p<0.05) than surface delivery for all concentrations of E₂, except control, however surface delivery resulted in more statistically different induction responses. Columns labeled with same letter designate conditions not statistically different; all other comparisons, p<0.05.

FIG. 4 shows array fabrication with soft lithography techniques to pattern DNA-lipid complex deposition and transfection. A polydimethylsiloxane (PDMS) mold (A) was reversibly sealed to polystyrene slides (B), so that the holes in the mold, termed microwells, served as reservoirs for deposition of DNA complexes onto the polystyrene (C). After complex deposition in the microwells, the PDMS mold was peeled away from the polystyrene slide, which was then rinsed thoroughly. Rhodamine-labeled DNA complexes were immobilized on the slide in distinct regions, replicating the pattern of microwells in the PDMS mold (D-F). Transfection of MCF-7 cells seeded onto these arrays of patterned complexes on polystyrene slides was also confined to the patterns, as determined by GFP expression (G-I).

FIG. 5 shows bioluminescence imaging to detect dual-luciferase expression in an array format. Transfection of MCF-7 cells seeded onto arrays of complexes was assayed after 24 hours by sequentially adding the renilla and firefly luciferase substrates. The renilla substrate, VIVIREN (10 μM), was first added into the media and the array was imaged to determine pTK-rLUC expression (A). D-Luciferin (1 mM) was subsequently added to the same array, which was then imaged to acquire a dual signal (B). Firefly luciferase expression (pLUC) was determined by subtracting the VIVIREN signal from the signal obtained through imaging with the D-luciferin. When normalized, the firefly luciferase signal was 34±8 fold greater than the respective renilla expression.

FIG. 6 shows arrays used to monitor ERα induction of transcriptional activity. Complexes formed with different plasmids were immobilized in different spots of the array, as follows: 1. pLUC, 2. none, 3. pERE(3×)TK-ffLUC, 4. pERE(3×)TK-ffLUC and pTK-rLUC (2:1 ratio), and 5. pβGAL. Cells seeded on the arrays were treated with combinations of ethanol control (A-B), E₂ (C-D), or E₂ plus FUL (E-F). Dual-luciferase levels were analyzed 24 hours later with bioluminescence imaging, by first imaging with the renilla luciferase substrate, VIVIREN (A, C, E) and then imaging each array with D-luciferin, the firefly luciferase substrate (B, E, F). Induction of the ERE-regulated plasmid system was calculated by normalizing firefly luciferase expression to renilla luciferase expression (G). Firefly luciferase expression was determined by subtracting the VIVIREN signal from the signal obtained through imaging with the D-luciferin. For spots containing both the pERE(3×)TK-ffLUC and pTK-rLUC plasmids (column 4), E₂ statistically induced firefly luciferase expression 10-fold over control or FUL conditions, reported as a ratio of firefly to renilla luciferase (G). Columns labeled with same letter designate conditions not statistically different; all other comparisons, p<0.001.

FIG. 7 shows concentration response of E₂ on the ERE reporter plasmid system in an array format. For spots containing both the pERE(3×)TK-ffLUC and pTK-rLUC plasmids, increasing the concentration of E₂ statistically increased the induction of firefly luciferase expression, verifying a true concentration-response of E₂ in the induction of this plasmid system in an array format. Columns labeled with same letter designate conditions not statistically different; all other comparisons, p<0.05.

FIG. 8 shows phase imaging of 1.5% alginate beads (a) before MTT staining (control), (b and c) after MT′T staining after 24 and 48 hr., and (d and e) imaging of beads containing 1.5% alginate and 0.2% collagen stained after 24 and 48 hr.

FIG. 9 shows transfection efficiency for MCF-7 cells as a function of (a) N:P ratio and (b) DNA concentration as determined by beta-galactosidase staining.

FIG. 10 shows phase images of alginate beads containing MCF-7 cells transfected with beta-galactosidase. DNA Beta-gal staining of cells occurred 1, 2, and 3 days after initial incubation (a, b, and c). Transfected cells appear darkly stained.

FIG. 11 shows luciferase expression in array in terms of photon flux.

### DEFINITIONS

To facilitate an understanding of the invention, a number of terms are defined below.

As used herein, the term “transfection molecule” refers to a molecule that is transfected (e.g., via placid and/or viral means) into a host cell (e.g., within a transfection cell array described herein). The molecule transfected may be a nucleic acid that is of interest with respect to its ability to confer a change in the phenotype of the host cells (e.g., alter transcription, translation, cell cycle, etc.). The molecule transfected may refer to a nucleic acid sequence that varies from one portion of an array to the next. The molecule transfected may also include plasmid and/or viral sequence. Nucleic acid sequences that are transfected may be coding sequences for a protein, or for an RNA molecule (e.g., which is transcribed into an anti-sense RNA sequence, a ribozyme or double-stranded RNA, etc.), or a regulatory sequence (e.g., as part of a reporter construct), or other type of sequence.

As used herein, the terms “spot” and “transfection zone,” refers to the location within an array that cells are exposed to transfection molecules. The location may be within a 2-D array or 3-D array. Within a 2-D array, a “spot” or “transfection zone” generally refers to a location where a transfection molecule is immobilized to a substrate surface. Within a 3-D array, a “spot” or “transfection zone” refers generally to regions where polymeric-mediated delivery of transfection molecules to cells occurs.

As used herein, the term “gene transfer system” refers to any means of delivering a composition comprising a nucleic acid sequence to a cell or tissue. For example, gene transfer systems include, but are not limited to, vectors (e.g., retroviral, adenoviral, adeno-associated viral, and other nucleic acid-based delivery systems), microinjection of naked nucleic acid, polymer-based delivery systems (e.g., liposome-based and metallic particle-based systems), biolistic injection, and the like. As used herein, the term “viral gene transfer system” refers to gene transfer systems comprising viral elements (e.g., intact viruses, modified viruses and viral components such as nucleic acids or proteins) to facilitate delivery of a sample (e.g., a nucleic acid encoding a fusion protein of the present invention) to a desired cell or tissue. As used herein, the term “adenovirus gene transfer system” refers to gene transfer systems comprising intact or altered viruses belonging to the family Adenoviridae.

As used herein, the term “site-specific recombination target sequences” refers to nucleic acid sequences that provide recognition sequences for recombination factors and the location where recombination takes place.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N-6-methyladenosine, aziridinylcytosine, pseudoisocytosine, 5-(carboxyhydroxylmethyl)uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxy-aminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, RNA (e.g., including but not limited to, mRNA, tRNA and rRNA) or precursor. The polypeptide, RNA, or precursor can be encoded by a full length coding sequence or by any portion thereof. The term also encompasses the coding region of a structural gene and the sequences located adjacent to the coding region on both the 5′ and 3′ ends for a distance of about 1 kb on either end such that the gene corresponds to the length of the full-length mRNA. The sequences that are located 5′ of the coding region and which are present on the mRNA are referred to as 5′ untranslated sequences. The sequences that are located 3′ or downstream of the coding region and that are present on the mRNA are referred to as 3′ untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

Where “amino acid sequence” is recited herein to refer to an amino acid sequence of a naturally occurring protein molecule, “amino acid sequence” and like terms, such as “polypeptide” or “protein” are not meant to limit the amino acid sequence to the complete, native amino acid sequence associated with the recited protein molecule.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

The term “wild-type” refers to a gene or gene product that has the characteristics of that gene or gene product when isolated from a naturally occurring source. A wild-type gene is that which is most frequently observed in a population and is thus arbitrarily designed the “normal” or “wild-type” form of the gene. In contrast, the terms “modified,” “mutant,” “polymorphism,” and “variant” refer to a gene or gene product that displays modifications in sequence and/or functional properties (i.e., altered characteristics) when compared to the wild-type gene or gene product. It is noted that naturally-occurring mutants can be isolated; these are identified by the fact that they have altered characteristics (e.g., increased or decreased solubility) when compared to the wild-type gene or gene product.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc.). Heterologous genes are distinguished from endogenous genes in that the heterologous gene sequences are typically joined to DNA sequences that are not found naturally associated with the gene sequences in the chromosome or are associated with portions of the chromosome not found in nature (e.g., genes expressed in loci where the gene is not normally expressed).

DNA molecules are said to have “5′ ends” and “3′ ends” because mononucleotides are reacted to make oligonucleotides or polynucleotides in a manner such that the 5′ phosphate of one mononucleotide pentose ring is attached to the 3′ oxygen of its neighbor in one direction via a phosphodiester linkage. Therefore, an end of an oligonucleotides or polynucleotide, referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide or polynucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements that direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

As used herein, the terms “an oligonucleotide having a nucleotide sequence encoding a gene” and “polynucleotide having a nucleotide sequence encoding a gene,” means a nucleic acid sequence comprising the coding region of a gene or, in other words, the nucleic acid sequence that encodes a gene product. The coding region may be present in a cDNA, genomic DNA, or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the term “regulatory element” refers to a genetic element that controls some aspect of the expression of nucleic acid sequences. For example, a promoter is a regulatory element that facilitates the initiation of transcription of an operably linked coding region. Other regulatory elements include splicing signals, polyadenylation signals, termination signals, etc.

As used herein, the terms “complementary” or “complementarity” are used in reference to polynucleotides (i.e., a sequence of nucleotides) related by the base-pairing rules. For example, the sequence 5′-A-G-T-3′, is complementary to the sequence 3′-T-C-A-5′. Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids. Complementarity can include the formation of base pairs between any type of nucleotides, including non-natural bases, modified bases, synthetic bases and the like.

The term “homology” refers to a degree of complementarity. There may be partial homology or complete homology (i.e., identity). A partially complementary sequence is one that at least partially inhibits a completely complementary sequence from hybridizing to a target nucleic acid and is referred to using the functional term “substantially homologous.” The term “inhibition of binding,” when used in reference to nucleic acid binding, refers to inhibition of binding caused by competition of homologous sequences for binding to a target sequence. The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that lacks even a partial degree of complementarity (e.g., less than about 30% identity); in the absence of non-specific binding the probe will not hybridize to the second non-complementary target.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.).

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

A gene may produce multiple RNA species that are generated by differential splicing of the primary RNA transcript. cDNAs that are splice variants of the same gene will contain regions of sequence identity or complete homology (representing the presence of the same exon or portion of the same exon on both cDNAs) and regions of complete non-identity (for example, representing the presence of exon “A” on cDNA 1 wherein cDNA 2 contains exon “B” instead). Because the two cDNAs contain regions of sequence identity they will both hybridize to a probe derived from the entire gene or portions of the gene containing sequences found on both cDNAs; the two splice variants are therefore substantially homologous to such a probe and to each other.

When used in reference to a single-stranded nucleic acid sequence, the term “substantially homologous” refers to any probe that can hybridize (i.e., it is the complement of) the single-stranded nucleic acid sequence under conditions of low stringency as described above.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the T_{M} of the formed hybrid, and the G:C ratio within the nucleic acids.

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41 (% G+C), when a nucleic acid is in aqueous solution at 1 M NaCl (See e.g., Anderson and Young, Quantitative Filter Hybridization, in Nucleic Acid Hybridization (1985)). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. Those skilled in the art will recognize that “stringency” conditions may be altered by varying the parameters just described either individually or in concert. With “high stringency” conditions, nucleic acid base pairing will occur only between nucleic acid fragments that have a high frequency of complementary base sequences (e.g., hybridization under “high stringency” conditions may occur between homologs with about 85-100% identity, preferably about 70-100% identity). With medium stringency conditions, nucleic acid base pairing will occur between nucleic acids with an intermediate frequency of complementary base sequences (e.g., hybridization under “medium stringency” conditions may occur between homologs with about 50-70% identity). Thus, conditions of “weak” or “low” stringency are often required with nucleic acids that are derived from organisms that are genetically diverse, as the frequency of complementary sequences is usually less. “High stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 0.1×SSPE, 1.0% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 1.0×SSPE, 1.0% SDS at 42° C. when a probe of about 500 nucleotides in length is employed.

“Low stringency conditions” comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.1% SDS, 5×Denhardt's reagent (50×Denhardt's contains per 500 ml: 5 g Ficoll (Type 400, Pharamcia), 5 g BSA (Fraction V; Sigma)) and 100 μg/ml denatured salmon sperm DNA followed by washing in a solution comprising 5×SSPE, 0.1% SDS at 42° C. when a probe of about 500 nucleotides in length is employed.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.) (see definition above for “stringency”).

The term “fragment” as used herein refers to a polypeptide that has an amino-terminal and/or carboxy-terminal deletion as compared to the native protein, but where the remaining amino acid sequence is identical to the corresponding positions in the amino acid sequence deduced from a full-length cDNA sequence. Fragments typically are at least 4 amino acids long, preferably at least 20 amino acids long, usually at least 50 amino acids long or longer, and span the portion of the polypeptide required for intermolecular binding of the compositions (claimed in the present invention) with its various ligands and/or substrates.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring.

As used herein, the term “recombinant DNA molecule” as used herein refers to a DNA molecule that is comprised of segments of DNA joined together by means of molecular biological techniques.

As used herein, the term “antisense” is used in reference to RNA sequences that are complementary to a specific RNA sequence (e.g., mRNA). Included within this definition are antisense RNA (“asRNA”) molecules involved in gene regulation by bacteria. Antisense RNA may be produced by any method, including synthesis by splicing the gene(s) of interest in a reverse orientation to a viral promoter that permits the synthesis of a coding strand. Once introduced into an embryo, this transcribed strand combines with natural mRNA produced by the embryo to form duplexes. These duplexes then block either the further transcription of the mRNA or its translation. In this manner, mutant phenotypes may be generated. The term “antisense strand” is used in reference to a nucleic acid strand that is complementary to the “sense” strand. The designation (−) (i.e., “negative”) is sometimes used in reference to the antisense strand, with the designation (+) sometimes used in reference to the sense (i.e., “positive”) strand.

As used herein the term “coding region” when used in reference to a structural gene refers to the nucleotide sequences that encode the amino acids found in the nascent polypeptide as a result of translation of a mRNA molecule. The coding region is bounded, in eukaryotes, on the 5′ side by the nucleotide triplet “ATG” that encodes the initiator methionine and on the 3′ side by one of the three triplets, which specify stop codons (i.e., TAA, TAG, TGA).

As used herein the term “portion” when in reference to a nucleotide sequence (as in “a portion of a given nucleotide sequence”) refers to fragments of that sequence. The fragments may range in size from four nucleotides to the entire nucleotide sequence minus one nucleotide (e.g., 10 nucleotides, 20, 30, 40, 50, 100, 200, etc.).

As used herein, the terms “restriction endonucleases” and “restriction enzymes” refer to bacterial enzymes, each of which cut double-stranded DNA at or near a specific nucleotide sequence.

The terms “in operable combination,” “in operable order,” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced. The present invention is not limited to naturally occurring protein molecules. For example, the present invention contemplates synthesis of fusion proteins comprising multiple regions of unique polypeptide sequences (e.g., a Tat leader sequence, a target protein sequence, and marker protein sequence).

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids are nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

As used herein, the term “native protein” is used to indicate that a protein does not contain amino acid residues encoded by vector sequences; that is, the native protein contains only those amino acids found in the protein as it occurs in nature. A native protein may be produced by recombinant means or may be isolated from a naturally occurring source.

As used herein the term “portion” when in reference to a protein (as in “a portion of a given protein”) refers to fragments of that protein. The fragments may range in size from four amino acid residues to the entire amino acid sequence minus one amino acid.

The term “Southern blot,” refers to the analysis of DNA on agarose or acrylamide gels to fractionate the DNA according to size followed by transfer of the DNA from the gel to a solid support, such as nitrocellulose or a nylon membrane. The immobilized DNA is then probed with a labeled probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists (J. Sambrook et al., Molecular Cloning: A Laboratory Manual, Cold Spring Harbor Press, NY, pp 9.31-9.58 (1989)).

The term “Northern blot,” as used herein refers to the analysis of RNA by electrophoresis of RNA on agarose gels to fractionate the RNA according to size followed by transfer of the RNA from the gel to a solid support, such as nitrocellulose or a nylon membrane. The immobilized RNA is then probed with a labeled probe to detect RNA species complementary to the probe used. Northern blots are a standard tool of molecular biologists (J. Sambrook, et al., supra, pp 7.39-7.52 (1989)).

The term “Western blot” refers to the analysis of protein(s) (or polypeptides) immobilized onto a support such as nitrocellulose or a membrane. The proteins are run on acrylamide gels to separate the proteins, followed by transfer of the protein from the gel to a solid support, such as nitrocellulose or a nylon membrane. The immobilized proteins are then exposed to antibodies with reactivity against an antigen of interest. The binding of the antibodies may be detected by various methods, including the use of radiolabeled antibodies.

The term “transgene” as used herein refers to a foreign gene that is placed into an organism by, for example, introducing the foreign gene into newly fertilized eggs or early embryos. The term “foreign gene” refers to any nucleic acid (e.g., gene sequence) that is introduced into the genome of an animal by experimental manipulations and may include gene sequences found in that animal so long as the introduced gene does not reside in the same location as does the naturally occurring gene.

As used herein, the term “vector” is used in reference to nucleic acid molecules that transfer DNA segment(s) from one cell to another. The term “vehicle” is sometimes used interchangeably with “vector.” Vectors are often derived from plasmids, bacteriophages, or plant or animal viruses.

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

The terms “overexpression” and “overexpressing” and grammatical equivalents, are used in reference to levels of mRNA to indicate a level of expression approximately 3-fold higher (or greater) than that observed in a given tissue in a control or non-transgenic animal. Levels of mRNA are measured using any of a number of techniques known to those skilled in the art including, but not limited to Northern blot analysis. Appropriate controls are included on the Northern blot to control for differences in the amount of RNA loaded from each tissue analyzed (e.g., the amount of 28S rRNA, an abundant RNA transcript present at essentially the same amount in all tissues, present in each sample can be used as a means of normalizing or standardizing the mRNA-specific signal observed on Northern blots). The amount of mRNA present in the band corresponding in size to the correctly spliced transgene RNA is quantified; other minor species of RNA which hybridize to the transgene probe are not considered in the quantification of the expression of the transgenic mRNA.

The term “transfection” as used herein refers to the introduction of foreign DNA into eukaryotic cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

The term “calcium phosphate co-precipitation” refers to a technique for the introduction of nucleic acids into a cell. The uptake of nucleic acids by cells is enhanced when the nucleic acid is presented as a calcium phosphate-nucleic acid co-precipitate. The original technique of Graham and van der Eb (Graham and van der Eb, Virol., 52:456 (1973)), has been modified by several groups to optimize conditions for particular types of cells. The art is well aware of these numerous modifications.

The term “stable transfection” or “stably transfected” refers to the introduction and integration of foreign DNA into the genome of the transfected cell. The term “stable transfectant” refers to a cell that has stably integrated foreign DNA into the genomic DNA.

The term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectant” refers to cells that have taken up foreign DNA but have failed to integrate this DNA.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used herein, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The term “candidate agent” may be any substance that potentially inhibits or enhances protein folding and/or solubility, including, but not limited to, any chemical entity, pharmaceutical, drug, and the like (e.g., a small molecule or compound). Candidate agents may include fragments or parts of naturally-occurring proteins or compounds, or may be found as active combinations of known proteins or compounds, which are otherwise inactive. It is to be understood that candidate agents comprise both known and potential solubility inhibiting or enhancing agents. A candidate agent can be determined to be capable of altering target protein solubility and/or folding using the methods of the present invention.

As used herein, the term “host cell” refers to any cell, whether located in vitro or in vivo, that can be, or has been, a recipient for or incorporates exogenous nucleic acid sequences (e.g., vectors comprising fusion protein sequence), polynucleotides and/or proteins of the present invention. It is also meant to include progeny of a single cell, and the progeny may not necessarily be completely identical (e.g., in morphology or in genomic or total DNA complement) to the original parent cell due to natural, accidental, or deliberate mutations. The cells may be eukaryotic or prokaryotic and include, but are not limited to bacterial cells (e.g., E. coli) yeast cells, mammalian cells, avian cells, amphibian cells, plant cells, fish cells, and insect cells).

### DETAILED DESCRIPTION OF THE INVENTION

Analysis of multiple pathways or genes in a parallel format can be achieved using a transfected cell array, a high throughput technique to correlate gene expression with functional cell responses, based on gene delivery from a substrate that supports cell adhesion (See, e.g., Bengali et al., 2005, Biotechnol Bioeng 90(3):290-302; Pannier et al., 2005, Acta Biomaterialia 1(5):511-522; Segura and Shea, 2002, Bioconjug Chem 13(3):621-9; and Segura et al., 2003, J Control Release 93(1):69-84). While traditional microarrays can quantify the expression level of thousands of genes, they cannot accurately describe the functional activity of these genes in a cellular and physiological context (See, e.g., Pepperkok and Ellenberg, 2006, Nat Rev Mol Cell Biol 7(9):690-6). Transfected cell arrays present an approach to study gene function in the context of a living cell, allowing proteins to be translated and folded correctly and to interact within the environment of the cell. Additionally, a large number of genes can potentially be screened in parallel for induction or repression of a given function (See, e.g., Palmer and Freeman, 2005, Pharmacogenomics 6(5):527-34). Transfected cell arrays initially offered the hope of compact, economical, and high-throughput analysis in living cells (See, e.g., Hook et al., 2006, Trends Biotechnol 24(10):471-7; Palmer and Freeman, 2005, Pharmacogenomics 6(5):527-34).

Since the original report on transfected cell arrays (See, e.g., Ziauddin and Sabatini, 2001, Nature 411(6833):107-10), reverse transfection has been employed in several high-throughput cell based microarrays to screen for gene function or activity. Reverse transfection involves printing mixtures of different transfection molecules (e.g., viruses and/or plasmids) with or without a carrier (e.g., gelatin) into specific domains onto a substrate. Next, a lipid-based transfection agent can be floated over the array, and cells subsequently seeded to form a living cell microarray of locally transfected cells in a lawn of nontransfected cells. The first transfected cell array was used to analyze genes for phosphotyrosine activity and identified six genes; five genes that encode known tyrosine kinase proteins and one that encodes a protein of unknown function (See, e.g., Ziauddin and Sabatini, 2001, Nature 411(6833):107-10). Transfected cell arrays have since been applied to study signaling pathways (See, e.g., Webb et al., 2003, J Biomol Screen 8(6):620-3; to screen antibody fragments (See, e.g., Delehanty et al., 2004, Anal Chem 76(24):7323-8); identify possible new lysophosphatidic acid receptors (See, e.g., Lee et al., 2006, J Biol Chem 281(33):23589-97; perform protein localization studies (See, e.g., Hu et al., 2005, Biochem Soc Trans 33(Pt 6):1407-8; Hu et al., 2006, BMC Genomics 7:155), screen for proapoptotic genes (See, e.g., Mannherz et al., 2006, Genomics 87(5):665-72); Palmer et al., 2006, BMC Genomics 7(1):145), and annotate protein function (See, e.g., Hodges et al., Mol Cell Proteomics 4(9): 1319-27). The transfected cell array has also been adapted to high-throughput RNAi studies (See, e.g., Mousses et al., Genome Res 13(10):2341-7), for example, for the analysis of spindle formation (See, e.g., Silva et al., 2004, Proc Natl Acad Sci USA 101(17):6548-52), secretory pathways (Erfle et al., 2004, Biotechniques 37(3):454-8, 460, 462), and chromosome segregation and nuclear structure in a time-lapse system (See, e.g., Neumann et al., 2006, Nat Methods 3(5):385-90).

Some technological improvements have enhanced the capabilities of the arrays, but have failed to yield widespread application of transfection cell arrays. Most efforts have focused on increasing transfection efficiency within the array by using preformed complexes (See, e.g., Delehanty et al., 2004, Biosens Bioelectron 20(4):773-9; Delehanty et al., 2004, Anal Chem 76(24):7323-8; Erfle et al., 2004, Biotechniques 37(3):454-8, 460, 462; Hodges et al., 2005, Mol Cell Proteomics 4(9):1319-27; Mousses et al., 2003, Genome Res 13(10):2341-7; Pannier et al., 2005, Acta Biomaterialia 1(5):511-522; Redmond et al., 2004, Mol Cell Proteomics 3(8):770-9; Silva et al., 2004, Proc Natl Acad Sci USA 101(17):6548-52; and Yoshikawa et al, 2004, J Control Release 96(2):227-32); incorporating fibronectin (See, e.g., Yoshikawa et al, 2004, J Control Release 96(2):227-32); atelocollagen (See, e.g., Honma et al., 2001, Biochem Biophys Res Commun 289(5):1075-81); and recombinant proteins (See, e.g., Redmond et al., 2004, Mol Cell Proteomics 3(8):770-9), with plasmid or DNA complexes, manipulating substrate hydrophobicity (See, e.g., Delehanty et al., 2004, Biosens Bioelectron 20(4):773-9), or coating cationic polymer and collagen onto surfaces prior to transfection (See, e.g., Chang et al., 2004, Nucleic Acids Res 32(3):e33). Micropatterning strategies have also been used to fabricate arrays, using self-assembled monolayers to pattern DNA (See, e.g., Pannier et al., 2005, Acta Biomaterialia 1(5):511-522; Yamauchi et al., 2004, Biochim Biophys Acta 1672(3):138-47) or siRNA (See, e.g., Fujimoto et al., 2006, Bioconjug Chem 17(6):1404-10) complex immobilization on gold slides or electrodes (See, e.g., Yamauchi et al., 2004, Nucleic Acids Res 32(22):e187; Yamauchi et al., 2005, Langmuir 21(18):8360-7). Arrays have been formed with dendrimers (See, e.g., How et al., 2004, Comb Chem High Throughput Screen 7(5):423-30) and viral vectors (See, e.g., Bailey et al., 2006, Nat Methods 3(2): 117-22; Hobson et al., 2003, BMC Biotechnol 3(1):4; and Michiels et al., 2002, Nat Biotechnol 20(11):1154-7) for enhanced gene delivery, magnetic beads (Isalan et al., 2005, Nat Methods 2(2): 113-8) or hydrogels (See, e.g., Peterbauer et al., 2006, Lab Chip 6(7):857-63) to localize cells and vectors, and for alternative cell types, including Drosophila (See, e.g., Wheeler et al., 2004, Nat Methods 1(2):127-32) and non-adherent cells (Kato et al., 2004, Biotechniques 37(3):444-8, 450, 452).

However, multiple problems remain that have precluded widespread application of transfection cell arrays. These problem include poor transfection efficiency, spot-to-spot variability, an inability to normalize data, complex and time consuming post-transfection processing, lack of sensitivity, poor image acquisition and quantification, as well as limitations prohibiting expansion of assessed biological endpoints.

Moreover, while there has been much interest in two dimensional transfected cell arrays (e.g., see references cited above), three-dimensional transfected cell arrays have heretofore not been described.

Accordingly, in some embodiments, the present invention relates to characterizing transcription within cells. In particular, the present invention provides transfected cell arrays (e.g., two-dimensional and/or three-dimensional arrays) and systems, kits and methods utilizing the same (e.g., for transcriptional activity characterization). Compositions and methods of the present invention find use in, among other things, research, drug discovery and clinical (e.g., diagnostic, preventative and therapeutic) applications.

In some embodiments, the present invention provides compositions and methods for generating transfection cell arrays. In some embodiments, the present invention provides a transfection cell array that allows for normalization of transfection and provides rapid and sensitive methods for quantification of cellular responses with minimal post-transfection processing.

For example, in some embodiments, the present invention provides a plurality (e.g., two or more) plasmid system that provide normalization, sensitivity, and quantification (e.g., of transfection efficiency and cellular responses). Previously utilized transfection cell arrays have been described as suffering from spot to spot variability in transfection that compromises the ability to quantify a response within an array (e.g., because sub-maximal responses may indicate either a limited effect or simply inefficient or unequal delivery). Additionally, a variance in fluorescence intensity of transfected cells (e.g., using GFP) has been noted between spots of arrays (See, e.g., Hook et al., 2006, Trends Biotechnol 24(10):471-7). Accordingly, the present invention addresses these shortcoming by providing, in some embodiments, compositions and methods that enable normalization of transfection efficiency. For example, in some embodiments, a plurality plasmid (e.g., two or more plasmid) system comprising a normalization plasmid (e.g., comprising a reporter molecule (e.g., luciferase reporter)) that is present within each spot (e.g., transfection zone), and a functional plasmid (e.g., comprising a reporter molecule (e.g., luciferese reporter (e.g., that is the same or different than that within a normalization plasmid))) that varies between spots and is responsible for the functional endpoint of the array, is deposited in each spot/transfection zone. The present invention is not limited by the number or type of plasmids utilized in a system. Indeed, two, three, four or more plasmids (e.g., comprising similar or different components) may each be present in a transfection zone or spot. In some embodiments, the plurality of plasmids each contain the same promoter (e.g., TK promoter or other promoter described herein). In some embodiments, the presence of the same promoter permits normalization and/or allows comparison between one or more cell lines on and/or in an array (e.g., a 2-dimensional or 3-dimensional array, respectively).

In some embodiments, delivery of a plurality of plasmids results in a majority of cells expressing two or more reporter genes (e.g., one from each plasmid). In order to normalize data (e.g., with a second, third, fourth or more plasmid), efficiency of delivery should be sufficient to obtain a signal from each plasmid. The present invention addresses obstacles encountered with poor cellular transfection of multiple plasmids in previously described transfection cell arrays (e.g., that utilized small transfection zone and/or spot sizes (See, e.g., Palmer and Freeman, 2005, Pharmacogenomics 6(5):527-34)). For example, small spot/transfection zone size are detrimental because each spot on the array may contain so few cells that an insufficient number of cells are transfected locally to be statistically informative (See, e.g., Hodges et al., 2005, Mol Cell Proteomics 4(9):1319-27). Small spots/transfection zones with low transfection efficiency make image acquisition and quantification difficult and less sensitive, leading to high false positive and false negative rates (See, e.g., Palmer et al., 2006, BMC Genomics 7(1):145). Thus, in some embodiments, the present invention utilizes spot sizes that are larger relative to traditional transfection cell arrays. For example, in some embodiments the present invention provides a method of generating a 2-D transfection cell array comprising forming holes in a first material (e.g., polydimethylsiloxane (PDMS) or other material described herein) to generate an array mold, attaching the array mold to a solid substrate (e.g., glass, plastic, or other substrate material disclosed herein) to form a complex comprising a plurality of wells, depositing one or more transfection molecules (e.g., nucleic acid (e.g., RNA, DNA, etc. (e.g., within a plasmid or virus)) into the plurality of wells under conditions such that the transfection molecules are immobilized on the solid substrate (e.g., in the hole (e.g., spot or transfection zone)) and removing the array mold from the solid substrate to generate an array (e.g., of one or more transfection molecules) on the substrate. In some embodiments, the transfection molecules comprise a nucleic acid vector (e.g., plasmids). In some embodiments, the plasmid comprises a reporter gene. In some embodiments, the plasmid comprises a promoter region (e.g., configured to bind transcription factors). In some embodiments, the solid substrate is polystyrene. In some embodiments, the spot or transfection zone comprises a second vector (e.g., plasmid). In some embodiments, a second plasmid comprises a nucleic acid sequence comprising a reporter gene and a promoter region (e.g., that permits transcription efficiency to be normalized (e.g., between spots/transfections zones present in the array)). Preferred sizes of the spots/transfection zones are described herein. In some embodiments, a transfection cell array of the present invention (e.g., fabricated by methods disclosed herein (e.g., to produce large, useful spot/transfection zone sizes) is compatible with viral delivery of transfection molecules (See, e.g., Bailey et al., 2006, Nat Methods 3(2):117-22; Hobson et al., 2003, BMC Biotechnol 3(1):4; Michiels et al., 2002, Nat Biotechnol 20(11):1154-7). In some embodiments, a transfection cell array of the present invention (e.g., fabricated by methods disclosed herein (e.g., to produce large, useful spot/transfection zone sizes) is compatible with plasmid delivery of transfection molecules. In some embodiments, a transfection cell array of the present invention is compatible with delivery of transfection molecules and/or other agents (e.g., candidate agents, drugs, oligonucleotides, etc.) via non-viral mediated and non-transfection mediated delivery (e.g., via receptor mediated endocytosis, phagocytosis, diffusion, etc.).

In some embodiments, the present invention provides a method of screening transcriptional activity within cells comprising exposing an array (e.g., of transfection zones on a solid substrate comprising transfection molecules (e.g., wherein the transfection molecules comprise a first nucleic acid vector comprising a reporter gene and a promoter region configured to bind transcription factors and a second nucleic acid vector comprising a promoter configured for standardization of transfection efficiency)), wherein the array is generated by forming holes in a first material (e.g., polydimethylsiloxane (PDMS) or other material described herein) to generate an array mold, attaching the array mold to a solid substrate (e.g., glass, plastic, or other substrate material disclosed herein) to form a complex comprising a plurality of wells, depositing one or more transfection molecules (e.g., nucleic acid (e.g., RNA, DNA, etc. (e.g., within a plasmid or virus)) into the plurality of wells under conditions such that the transfection molecules are immobilized on the solid substrate (e.g., in the hole (e.g., spot or transfection zone)) and removing the array mold from the solid substrate to generate an array (e.g., of one or more transfection molecules) on the substrate), to cells under conditions such that transcriptional activity within the cells is altered, and detecting the transcriptional activity within the cells. In some embodiments, detecting transcriptional activity within the cells comprises detecting expression of a reporter gene.

In some embodiments, bioluminescence imaging (See, e.g., Rutter et al., 1998, Chem Biol 5(11):R285-90) is used with a transfection cell array (e.g., 2-dimensional (2-D) and/or 3-dimensional (3-D) array) described herein to quantify the response of transfection molecules (e.g., two or more vectors carrying transfection molecules) within the array (e.g., with minimal post-transfection processing and with high sensitivity). Previously utilized transfection cell arrays suffer from endpoint analysis that may require tagging or staining (See, e.g., Hook et al., 2006, Trends Biotechnol 24(10):471-7) to report gene function, which can require extensive post-transfection processing, such as fixation and immunostaining (See, e.g., Lee et al., 2006, J Biol Chem 281(33):23589-97; Wheeler et al., 2005, Nat Methods 1(2):127-32). In contrast, a transfection cell array fabricated and used according to compositions and methods disclosed herein permit rapid endpoint quantification (e.g., in each spot/transfection zone (e.g., via sequential addition of substrates)). For example, in some embodiments, two or more transfection molecules (e.g., normalization and/or functional plasmid vectors) contain renilla and/or firefly luciferase reporters (e.g., the substrates of which can be added directly to culture media followed by imaging of the array (e.g., of cells present in the array (e.g., cells that have been transfected using compositions and methods of the present invention))). Luciferase reporters are more sensitive than GFP, without the issues of autofluorescence and background signals (See, e.g., Rutter et al., 1998, Chem Biol 5(11):R285-90). Luciferase is more quantitative and allows for small differences in expression to be determined, which, in some embodiments, permits an array of the present invention to determine a dose response to an external stimulus. In some embodiments, the short half-life of luciferase permits for real-time imaging (e.g., to identify and/or characterize dynamics of gene activity (See, e.g., Rutter et al., 1998, Chem Biol 5(11):R285-90). In some embodiments, the present invention utilizes imaging systems that use automated microscopy and image processing (See, e.g., Pepperkok and Ellenberg, 2006, Nat Rev Mol Cell Biol 7(9):690-6; Wheeler et al, 2005, Nat Methods 1(2):127-32) (e.g., that allow for detection of changes in cellular morphology and cellular expression (e.g., nucleic acid, protein, etc.) level data (e.g., that is not possible with bioluminescence imaging)). The present invention is not limited by the endpoint data analysis methods utilized. Indeed, a variety of other methods may find use with the compositions and methods of the present invention including those described herein.

Compositions and methods of the present invention were utilized to quantify the activity of estrogen rector-alpha (ERα) in breast cancer cells with an estrogen-response-element (ERE)-regulated promoter reporter system (e.g., as an example of an inducible vector (e.g., plasmid) system (e.g., in a cancer model)). ERα, a member of the nuclear receptor superfamily of transcription factors, activates transcription through binding of its ligand, 17-β-estradiol (E₂). Expression of ERα is used clinically as a biomarker to determine treatment for breast cancer patients (See, e.g., Ariazi et al., 2006, Curr Top Med Chem 6(3):181-202; Pearce and Jordan, 2004, Crit. Rev Oncol Hematol 50(1):3-22). However, simple expression of transcription factors like ERα does not necessarily reflect pathway activation, as transcription factor activity is regulated through diverse mechanisms (See, e.g., Levine and Tijan, 2003, Nature 424(6945):147-51), including heteromeric complexes, ubiquitination, methylation, acetylation, and post-translational modifications such as phosphorylation. However, in some embodiments, compositions and methods of the present invention allow for the determination of transcription factor activity. For example, the present invention provides the ability to characterize the induction of activity of ERα by E₂. Induction in a transfection cell array of the present invention mimicked results obtained through traditional luciferase assay methods, with E₂ inducing luciferase expression 10-fold over fulvestrant or vehicle controls (See, e.g., Examples 1-5). An array of the present invention is also able to characterize activity in response to a range of dosages (e.g., the varying ER activity in response to a range of E₂ dosages was detected (See, e.g. Examples 4-5)) demonstrating the sensitivity of the compositions and methods disclosed herein.

In some embodiments, the present invention provides a 3-D array (e.g., transfection cell array). In some embodiments, the 3-D array is generated by spotting a substrate with a biopolymer solution containing cells (e.g., from a cell culture line, freshly harvested cells, cloned cells, transformed cells, etc.) and transfection molecules (e.g., vectors containing nucleic acid (e.g., DNA, RNA, cDNA, tRNA, siRNA) of interest) (See, e.g., Example 6). Thus, in some embodiments, the present invention provides a composition comprising a 3-D transfection cell array comprising an array of spatially controllable immobilized gels, wherein the gels comprise a crosslinkable biopolymer solution comprising cells exposed to transfection molecules (e.g., presented by a transfection reagent). Thus, in some embodiments, in contrast to substrate-mediated delivery of a transfection molecule (e.g., delivered via a 2-D transfected cell array (e.g., with a transfection molecule immobilized to a surface)), the present invention provides for polymeric-mediated delivery of transfection molecules to cells within a 3-dimensional transfection cell array (e.g., via distribution (e.g., homogenous distribution) of transfection molecules and cells within a polymer (e.g., biopolymer (e.g., acrylated poly(ethylene glycol) (PEG-Acryl)hydrogels, collagen gels, alginate gels, or other suitable biomaterial disclosed herein)).

In some embodiments, the biopolymer is cross-linked (e.g., forming an array of spatially controlled, immobilized gels). In some embodiments, the transcriptional activity of the cells (e.g., within spatially controlled, immobilized gels) is determined. The present invention is not limited by the method of characterizing the transcriptional activity of the cells. Indeed, a variety of methods are contemplated to be useful for characterizing such activity including, but not limited to those described herein. In some embodiments, a vector (e.g., plasmid) encoding a luciferase reporter gene driven by a regulated promoter is utilized. In some embodiments, expression of nucleic acid expression is determined/characterized. In some embodiments, detection of protein expression is determined/characterized. A vast of amount of information can be accumulated via such characterization. For example, the present invention provides a method for generating and/or characterizing information regarding signaling pathways and the production of genes relevant to cancer progression. Indeed, many other types of information are contemplated to be collected as described herein.

In some embodiments, transcriptional activation as well as transfection efficiency can be quantified (e.g., by analyzing the level of reporter (e.g., luciferase) expression (e.g., through automated and/or manual visual imaging of light production by the reporter (e.g., luciferase) upon the addition of its substrate luciferin)). In some embodiments, transfection efficiency is determined via normalization with a separate vector (e.g., plasmid) containing a minimal promoter driving a second reporter gene (e.g., a second luciferase or other reporter gene). In some embodiments, a 3-D transfection cell array provides an in vivo like cellular environment to evaluate cell performance. In some embodiments, the 3-D cellular array is utilized to evaluate (e.g., characterize and/or detect) cancer cell formation into cell structures. It is contemplated that data generated utilizing compositions and methods of the present invention will find use to assist in characterizing tumor stage and pathologic grade (e.g., physician's assessment of grade and/or pathology). However, the present invention is not so limited. For example, as described herein, information generated utilizing compositions and methods of the present invention also find use in molecular staging of disease, diagnostics and prognostics for disease and treatment, and as research tools (e.g., to analyze the activity of cellular signaling pathways (e.g., in specific cell populations and/or under specific environmental conditions), to characterize cellular matrix design (e.g., how matrix design correlates with cellular response and cellular signaling pathways), and for characterizing tissue engineering (e.g., by providing a method for screening changes in cell activity and morphology (e.g., as function of gel rigidity and composition (e.g., when various extracellular matrices are also incorporated)))). In some embodiments, 3-D transfection cell arrays and methods of using the same described herein provide information regarding effects on cell morphology and transcriptional activation as a function of gel elasticity, crosslink density, mesh size and/or composition (e.g., comprising extracellular matrices).

In some embodiments, the present invention provides compositions and methods (e.g., using 2-D and/or 3-D transfection cell arrays described herein) for high throughput analysis of gene function in cells. For example, in some embodiments, compositions and methods of the present invention provide the ability to screen large sets of nucleic acid constructs for those encoding desired products or for causing cellular phenotypes of interest. In some embodiments, cells transfected using a 2-D or 3-D cell array described herein can be screened for the ability of a transfection molecule (e.g., nucleic acid) and/or other agent (e.g., candidate agent) to confer a particular phenotype on the cells, and, by reference to the position of the cell(s) on the array, the identity of the transfection molecule (e.g., nucleic acid) or other agent can be determined.

Accordingly, the present invention is related to a method, referred to as a reverse transfection method, in which a defined transfection molecule (e.g., nucleic acid (e.g., a nucleic acid of known sequence or source)), is introduced into cells in defined areas (e.g., within a “lawn” of eukaryotic cells) in a 2-D array, or within a 3-D spatially controllable, immobilized gel comprising a crosslinkable biolpolymer cells and transfection molecules, in which the transfection molecules will be expressed or may have an effect on or interact with a cellular component or function. The present invention is not limited by the type of transfection molecule utilized. For example, any suitable nucleic acid such as an oligonucleotide, DNA and RNA can be used as a transfection molecule in the methods of the present invention. Moreover, the nucleic acid may be present in a plasmid or vector for expression, or not. It should be understood that any suitable nucleic acid can be utilized in the compositions and methods of the present invention.

In some embodiments, the present invention provides a method in which a transfection molecule (e.g., DNA (e.g., DNA of known sequence or source)) is introduced into cells in defined areas of a “lawn” of cells (e.g., eukaryotic cells), in which it will be expressed or will itself have an effect on or interact with a cellular component or function. In some embodiments, a mixture comprising transfection molecules (e.g., nucleic acid (e.g., DNA (e.g., cDNA)) of interest (e.g., incorporated into an expression plasmid or vector)) and a carrier protein (e.g., a lipid based or gelatin based carrier (See, e.g. U.S. Patent Application Publication No. 20030228601, hereby incorporated by reference in its entirety)) is deposited into a plurality of wells in an array mold (e.g., formed by generating holes in a first material (e.g., polydimethylsiloxane (PDMS) or other material described herein) to generate an array mold, attaching the array mold to a solid substrate (e.g., glass, plastic, or other substrate material disclosed herein) to form a complex comprising a plurality of wells)), under conditions such that the transfection molecules are immobilized on the solid substrate (e.g., in the hole (e.g., spot or transfection zone)), wherein the array mold is removed from the solid substrate to generate an array (e.g., of one or more transfection molecules) on the substrate. In some embodiments, a mixture comprising spatially controllable, immobilized gels, wherein the gels comprise a crosslinkable polymer, cells and transfection molecules are arrayed on a substrate to form a plurality of transfection zones or spots.

The transfection molecules and/or carrier protein(s) can be deposited in as many discrete locations (e.g., spots or transfection zones created by making holes in a first material (e.g., for 2-D arrays), or by controlling the spatial location of immobilized gels (e.g., for 3-D arrays)) as desired. Thus, the present invention is able to produce a surface bearing a plurality of transfection molecules in defined, distinct locations wherein the identity of the transfection molecule in each of the transfection zones/spots is known/defined.

In some embodiments, eukaryotic cells, such as mammalian cells (e.g., human, monkey, canine, feline, bovine, or murine cells), bacterial, insect, or plant cells, are plated (placed) onto the surface bearing the transfection molecules (e.g., vector DNA (e.g., for 2-D arrays)) or are mixed with transfection molecules and a crosslinkable linker (e.g., for 3-D arrays) in sufficient density and under appropriate conditions for introduction/entry of the transfection molecules into the eukaryotic cells (e.g., host cells) and expression of the DNA or its interaction with cellular components monitored and/or characterized.

In some embodiments, the concentration and/or amount of transfection molecules (e.g., placed in a hole formed in a first material (e.g., PDMS) or mixed with a crosslinkable polymer and cells can be determined empirically for each use. However, it is contemplated that when the transfection molecule is nucleic acid, the concentration will generally be in the range of from about 0.001 μg/μl to about 0.2 μg/μl and, in some embodiments, is from about 0.005 μg/μl to about 0.10 μg/μl. Alternatively, the concentration of DNA present in the mixture can be from about 0.01 μg/μl to about 0.5 μg/μl, from about 0.007 μg/μl to about 0.021 μg/μl and from about 0.01 μg/μl to about 0.3 μg/μl.

In some embodiments, the amount of nucleic acid utilized is determined per surface area. For example, in some embodiments, the amount of nucleic acid used will generally be in the range of from about 0.001 μg/cm² to about 5 μg/cm² and, in some embodiments, is from about 0.05 μg/cm² to about 0.5 μg/cm². Alternatively, the amount of DNA present can be from about 0.01 μg/cm² to about 1.0 μg/cm², from about 0.03 μg/cm²to about 0.75 μg/cm² and from about 0.1 μg/cm² to about 2.5 μg/cm².

In some embodiments, when a carrier molecule is utilized with transfection molecules for addition to a plurality of wells formed by punching holes in a first material (e.g., PDMS), the concentration of the carrier molecule (e.g., gelatin or another carrier macromolecule) can be determined empirically for each use, but will generally be in the range of 0.01% to 0.5% and, in specific embodiments, is from about 0.05% to about 0.5%, from about 0.05% to about 0.2% or from about 0.1% to about 0.2%.

In some embodiments, when cells are mixed with transfection molecules and a crosslinkable biopolymer (alginate and/or alginate/collagen), the concentration of the crosslinkable biopolymer can be determined empirically for each use, but will generally be in the range of 1% to 5% alginate, and, in specific embodiments, is from about 1% to about 2% alginate, from about 1.4% to about 1.6% alginate or from about 0.1% to about 5% alginate, although higher and lower percentages are contemplated. Similarly, the concentration of collagen will generally be in the range of 0.1% to 3% alginate, and, in specific embodiments, is from about 0.1% to about 0.3% alginate, from about 0.1% to about 0.2% alginate or from about 0.05% to about 5% alginate, although higher and lower percentages are contemplated.

In some embodiments, a charge neutralizing reagent (e.g., polyethyleimine (PEI)) is added to the solution comprising crosslinkable biopolymer and cells and transfection molecules. In some embodiments, the charge neutralizing reagent is added such that the nitrogen to phosphate (N:P) ratio is achieved to attain an optimal charge balance for transfection (e.g., optimal transfection). In some embodiments, the N:P ration is between around 15-35, from about 20-30, or from about 22-27, although higher and lower charge ratios may be used (e.g., depending on the type of cell and types of transfection molecules utilized). In some embodiments, a transfection efficiency of greater than about 50%, greater than 60%, greater than 70%, greater than 80%, or greater than 90% is achieved, although higher and lower transfection efficiencies may be attained.

In some embodiments, if the transfection molecule (e.g., nucleic acid (e.g., DNA, RNA, cDNA, etc.)) used is present in a vector, the vector can be of any type, such as a plasmid or viral-based vector, into which a nucleic acid of interest (e.g., DNA to be expressed in reverse transfected cells) can be introduced and expressed (e.g., after reverse transfection) in recipient cells. For example, a CMV-driven expression vector can be used. Commercially available plasmid-based vectors, such as pEGFP (CLONTECH) or pcDNA3 (INVITROGEN), or viral-based vectors can be used (See, e.g., U.S. Patent Application Publication Nos. 20030228601 and 20060257858, each of which is hereby incorporated by reference in its entirety for all purposes).

In some embodiments, to generate a transfection cell array, cells are either plated over an array formed on a solid surface (e.g., for a 2-D array) or are mixed with transfection molecules and a crosslinkable biopolymer prior to immobilizing in a spacially defined area on substrate (e.g., for a 3-D array). In some embodiments, actively growing cells are generally used and are plated, preferably in the range of 10⁴-10⁷ cells/cm² (although higher and lower concentrations are contemplated (e.g., depending upon the cell type) on top of the substrate surface containing the affixed transfection molecules (e.g., nucleic acids (e.g., in media such as Dulbecco's Modified Eagles Medium (DMEM) containing 10% heat-inactivated fetal serum (IFS) with L-glutamine and penicillin/streptomycin (pen/strep) or other media)); or for 3-D gels, are mixed with transfection molecules and a crosslinkable biopolymer in the range of 10⁴-10⁶ cells/mL, although more or less cells can also be used (e.g., based upon the type of cells to be transfected).

The transfection cell arrays (e.g., 2-D and/or 3-D arrays comprising cells and transfection molecules) are maintained under conditions appropriate for growth of the cells and entry of DNA, such as an entry of an expression vector containing the DNA, into cells. For example, in some embodiments, one to two cell cycles are sufficient for reverse transfection to occur, but this can vary depending upon the cell type and conditions used and the appropriate length of time for a specific combination can be determined empirically. After sufficient time has elapsed, cells can be assessed for reverse transfection (e.g., entry of transfection molecules (e.g., nucleic acid (e.g., DNA)) into cells) and expression of the encoded product or effect of the introduced DNA on reverse-transfected cells, using known methods. This can be done, for example, by detecting immunofluorescence or enzyme immunocytochemistry, autoradiography, in situ hybridization, or other means of detecting expression of the DNA or an effect of the encoded product or of the DNA itself on the cells into which it is introduced.

In some embodiments, detection of transfection (e.g., alteration of transcription within transfected cells) utilizes detection of a reporter system (e.g., renilla and/or firefly luciferase reporters (e.g., the substrates of which can be added directly to culture media followed by imaging of the array (e.g., of cells present in the array (e.g., cells that have been transfected using compositions and methods of the present invention))).

In some embodiments, immunofluorescence can used to detect expression of an encoded protein (e.g., an antibody that binds a protein and is fluorescently labeled can used (e.g., added to an array under conditions suitable for binding of the antibody to the protein) and the location (e.g., transfection zone or spot) of the protein identified by detecting fluorescence). In some embodiments, the presence of fluorescence indicates that reverse transfection has occurred and the encoded protein has been expressed in the defined location(s) which show fluorescence. The presence of a signal, detected by the method used, on the slides indicates that reverse transfection of the DNA into cells and expression of the encoded product or an effect of the DNA in recipient cells has occurred in the defined location(s) at which the signal is detected. As described above, the identity of the DNA present at each of the defined locations is known; thus, when expression occurs, the identity of the expressed protein is may also known.

In some embodiments, the present invention provides a method for expressing specific, identified nucleic acids (e.g., cDNAs or genomic DNAs), in defined locations or areas of a surface (e.g., transfection zones or spots) onto which the same or different nucleic acids (e.g., variants, mutants, etc.) have been attached. Thus, because each area of the surface can be covered/spotted with nucleic acid of a known composition, this permits identification of the expressed protein. In addition, the present method is useful to identify DNAs whose expression alters (enhances or inhibits) a pathway, such as a signaling pathway in a cell or another property of a cell, such as its morphology or pattern of gene expression. In some embodiments, compositions and methods of the present invention are useful, for example, as a high-throughput screening method, such as in a microarray format. Thus, compositions and methods described herein can be used for identifying nucleic acids whose expression change the post-translational status and/or subcellular location of a protein of interest or the effect of a test compound or agent on the cell (e.g., cellular activity post exposure to a test agent or compound (e.g., a small molecule, drug, hormone, siRNAetc.)). Thus, in some embodiments, compositions and methods of the present invention can be utilized to characterize cellular activity (e.g., transcriptional activity) post exposure of cells to a test agent, by measuring transcriptional activity (e.g., of a reporter system) within the cell.

In some embodiments, the present invention provides methods of making arrays of the present invention. The methods comprises The method comprises affixing DNAs or reverse transfected cells onto a surface by the steps described. herein for the gelatin-DNA embodiment or the lipid-DNA embodiment.

In some embodiments, the present invention relates to a method of introducing specific, known transfection molecules (e.g., nucleic acids (e.g., DNA)) at specific, discrete, defined locations (e.g., transfection zones or spots) on a substrate surface by means of a reverse transfection method. The size of the transfection zones/spots, the quantity (density) of the transfection zones/spots and the configuration of the transfection zone/spots can be adjusted depending upon a variety of factors (e.g., the type of cells used, the conditions used to transfect, etc.). For example, in some embodiments, the transfection zones/spots can be from about 0.1 to about 10 mm in diameter, from about 0.5 to about 10 mm in diameter, from about 1 to about 5 mm in diameter, from about 2 to about 4 mm in diameter, or from about 2.3 to about 2.5 mm in diameter, although larger and smaller diameters are contemplated. In some embodiments, each transfection zone/spot is from about 0.1 to about 0.9 mm away from another transfection zone/spot.

In some embodiments, the present invention provides identification and/or detection of cells into which transfection molecules have been transfected (e.g., reverse transfected). In one embodiment, transfection molecules (e.g., DNA) introduced into cells are expressed in the cells, either by an expression vector containing the molecules (e.g., DNA) or as a result of integration of molecules (e.g., DNA) into host cell DNA, from which it is expressed. In some embodiments, nucleic acids (e.g., RNA (e.g., siRNA) or DNA) are introduced to cells (e.g., are not transfectedly expressed in the cells), and are able to alter cellular components and/or function (e.g., that can be detected using the compositions and methods of the present invention (e.g., reporter constructs transfected into cells). For example, small inhibitory RNAs or other antisense molecules can be introduced into cells to alter cell function. In some embodiments, a nucleic acid can be transfected into the cell, wherein the nucleic acid inhibits expression of other nucleic acids in the cell. For example, a DNA fragment which is anti-sense to an mRNA (e.g., encoding a receptor for a drug) can be introduced into cells via reverse transfection. The anti-sense DNA may decrease the expression of mRNA (e.g., of the drug receptor protein (e.g., causing a decrease in drug binding to cells containing the anti-sense DNA)).

In some embodiments, the present invention provides detection of effects on recipient cells (cells containing transfection molecules introduced using an array of the present invention) that can be carried out by a variety of known techniques, such as immunofluorescence (e.g., of a reporter construct (e.g., a luciferase reporter) or of a protein (e.g., in which a fluorescently labeled antibody that binds a protein of interest (e.g., a protein thought to be encoded by a reverse transfected DNA or a protein whose expression or function is altered through the action of the reverse transfected DNA)).

In some embodiments, compositions and methods of the present invention are utilized to identify nucleic acids (e.g., DNAs) of interest (nucleic acids that are expressed in recipient cells or act upon or interact with recipient cell constituents or function (e.g., nucleic acids that encode a protein whose function is desired because of characteristics its expression gives cells in which it is expressed)). In some embodiments, an array of the present invention can be utilized as a protein or cell array (e.g., a protein microarray or a cell microarray).

The present invention is not limited by the type of nucleic acid utilized (e.g., as a transfection molecule (e.g., introduced into cells for expression)) in a 2-D and/or 3-D transfection array described herein. The nucleic acid molecules used in the transfection arrays of the present invention can be, for example, DNA, RNA or modified or hybrid forms thereof. The nucleic acids may be from any of a variety of sources, such as nucleic acid isolated from cells, or that which is recombinantly produced or chemically synthesized.

In some embodiments, the nucleic acids include coding sequence from cDNAs or genomic DNA. In addition to native sequences, the coding sequences can include those which have been mutated relative to the native sequence (e.g., a coding sequence that differs from a naturally occurring sequence by deletion, substitution or addition of at least one residue). It can correspond to full length or partial sequences, can be antisense in orientation, or can represent a non-coding sequence.

In some embodiments, all or a portion of the nucleic acid sequence can be synthesized chemically. Thus, random and semi-random sequence can be introduced into the nucleic acid sequences, as well as modified forms of nucleotides and nucleotide linkages, such as the use of modified backbones, methylated nucleotides and the like.

As described herein, the nucleic acid sequences can be present as part of a larger vector, such as an expression vector (e.g., a plasmid or viral-based vector), but it need not be. The nucleic acid of the array can be introduced into cells in such a manner that at least the a portion of the nucleic acid sequence (e.g., a portion that varies from transfection zone/spot to transfection zone/spot) becomes integrated into cellular genomic DNA and is expressed, or remains extrachromosomal (e.g., is maintained episomally).

The nucleic acid sequences (e.g., utilized in transfection cell arrays of the present invention) can be linear or circular, double stranded or single stranded, and can be of any size. In some embodiments (e.g., when expression vectors are used) nucleic acid sequences expressed in cells (e.g., resulting from transfection of cells with a vector comprising the nucleic acid sequence) is from about 200 nucleotides (nt) to about 10 kilobases (kb) in size, in some embodiments, from about 200 nt to about 5 kb, and in some embodiments, from about 200 nt to 2 kb, although shorter or longer sequences may be expressed. In some embodiments, the nucleic acid sequence transfected into cells (e.g., including vector sequence) is from about 1 kb to about 15 kb, although larger and smaller constructs are also contemplated herein.

In some embodiments, a transfection cell array (e.g., 2-D or 3-D array) of the present invention comprises a variegated library of expression vectors.

The present invention is not limited by the type of vector utilized (e.g., to introduce a transfection molecule (e.g., into cells for expression)) in a 2-D and/or 3-D transfection array described herein. In general, it will be desirable that the vector be capable of replication in the host cell. It may be a DNA (e.g., plasmid DNA) which is integrated into the host genome, and thereafter is replicated as a part of the chromosomal DNA, or it may be DNA which replicates autonomously (e.g., an episomal plasmid). In the latter case, the vector will include an origin of replication which is functional in the host. In the case of an integrating vector, the vector may include sequences that facilitate integration (e.g., sequences homologous to host sequences, or encoding integrases). The use of retroviral long terminal repeats (LTR) or adenoviral inverted terminal repeats (ITR) in the construct of a transfection array can, for example, facilitate the chromosomal integration of the construct.

Cloning and expression vectors for use with bacterial, fungal, yeast, and mammalian cellular hosts are known in the art, and are described in, for example, Powels et al. (Cloning Vectors: A Laboratory Manual, Elsevier, N.Y., 1985). Such vectors may be readily adapted for use in the present invention. The expression vectors may comprise non-transcribed elements such as an origin of replication, a suitable promoter and enhancer linked to the gene to be expressed, and other 5′ or 3′ flanking nontranscribed sequences, and 5′ or 3′ nontranslated sequences (e.g., ribosome binding sites, a poly-adenylation site, splice donor and acceptor sites, and transcriptional termination sequences).

In some embodiments, expression vectors contain both prokaryotic sequences (e.g., to facilitate the propagation of the vector in bacteria (e.g., in an amplification step after recovery from the array)), and/or one or more eukaryotic transcription units for expressing the target sequence in eukaryotic host cells. The pcDNAI/amp, pcDNAI/neo, pRc/CMV, pSV2gpt, pSV2neo, pSV2-dhfr, pTk2, pRSVneo, pMSG, pSVT7, PKO-neo and pHyg derived vectors are examples of mammalian expression vectors that can be readily adapted for use in the compositions and methods of the present invention. Some of these vectors are modified with sequences from bacterial plasmids, such as pBR322, to facilitate replication and drug resistance selection in both prokaryotic and eukaryotic cells. Alternatively, derivatives of viruses, such as the bovine papillomavirus (BPV-1), or Epstein-Barr virus (pHEBo, pREP-derived and p205) and the like, can also be used. The various methods employed in the preparation of the plasmids are well known in the art. For other suitable expression systems for both prokaryotic and eukaryotic cells, as well as general recombinant procedures, see, e.g., Molecular Cloning A Laboratory Manual, 2nd Ed., ed. by Sambrook, Fritsch and Maniatis (Cold Spring Harbor Laboratory Press: 1989) Chapters 16 and 17.

In some embodiments, vectors contain regulatory elements that can be linked to the target sequence for transfection of mammalian cells. Examples of such vectors include, but are not limited to, cytomegalovirus (CMV) promoter-based vectors such as pcDNA1 (INVITROGEN, San Diego, Calif.), MMTV promoter-based vectors such as pMAMNeo (CLONTECH, Palo Alto, Calif.) and pMSG (PHARMACIA, Piscataway, N.J.), and SV40 promoter-based vectors such as pSVO (CLONTECH, Palo Alto, Calif.).

A number of vectors exist for the expression of recombinant proteins in yeast (e.g., where yeast are used as a host cell in an array). Examples include, but are not limited to, YEP24, YIP5, YEP51, YEP52, pYES2, and YRP17, and other cloning and expression vehicles useful in the introduction of genetic constructs into S. cerevisiae (See, e.g., Broach et al. (1983) in Experimental Manipulation of Gene Expression, ed. M. Inouye Academic Press, p. 83, incorporated by reference herein). These vectors can replicate in E. coli due the presence of the pBR322 ori, and in S. cerevisiae due to the replication determinant of the yeast 2 micron plasmid. Moreover, if yeast are used as a host cell, it will be understood that the expression of a gene in a yeast cell requires a promoter which is functional in yeast. Suitable promoters include, but are not limited to, the promoters for metallothionein, 3-phosphoglycerate kinase (See, e.g., Hitzeman et al., J. Biol. Chem. 255, 2073 (1980) or other glycolytic enzymes (See, e.g., Hess et al., J. Adv. Enzyme Req. 7, 149 (1968); and Holland et al. Biochemistry 17, 4900 (1978)), such as enolase, glyceraldehyde-3-phosphate dehydrogenase, hexokinase, pyruvate decarboxylase, phospho-fructokinase, glucose-6-phosphate isomerase, 3-phosphoglycerate mutase, pyruvate kinase, triosephosphate isomerase, phospho-glucose isomerase, and glucokinase.

The present invention is not limited by the source of transfection moleclue (e.g., introduced into cells for expression)) utilized in a 2-D and/or 3-D transfection array described herein. For example, in some instances, it may be desirable to derive the host cell using insect cells. In such embodiments, the transfection array can be derived from, for example, a baculovirus expression system. Examples of such baculovirus expression systems include, but are not limited to, pVL-derived vectors (e.g., pVL1392, pVL1393 and pVL941), pAcUW-derived vectors (e.g., pAcUWI), and pBlueBac-derived vectors (e.g., beta-galactosidase containing pBlueBac III).

In some embodiments, where the source of target sequence for the array are naturally occurring, sequences can be isolated from any cell or collection of cells. For example, the target sequences can be isolated from the cells of either adult tissue or organs or embryonic tissue or organs at any given developmental stage (e.g., including, but not limited to, oocyte, blastocyte, etc.). The cells can be derived from healthy tissue or diseased tissue. In the case of a solid organ, cells can be obtained by biopsy or other mechanical means. For blood, lymph and other bodily fluids, cells can be isolated from the fluid component (e.g., by filtration, affinity purification, centrifugation or any other technique known in the art). The cells can be isolated to include a specific subset of phenotypes of cells from a given tissue, or can include or can be derived to include all or a substantial portion of cells representative of the tissue. For example, cells can be derived from an organ where the cells are particularly of epithelial, mesenchymal or endothelial origin. Subsets of cells can be isolated, for example, by use of cell surface markers or careful sectioning of a tissue.

In some embodiments, cells can be harvested from neoplastic or cancerous tissue. For example, cells may be cultured from any type of tumor or cancerous tissue (e.g., identified via pathologic means).

In some embodiments, nucleic acid sequences (e.g., transfected into host cells) are cDNA sequences (e.g., derived from mRNA isolated from a cell or cells of interest (e.g., cancer cell or normal cell) or that are chemically synthesized). There are a variety of methods known in the art for isolating RNA from a cellular source. For example, isolation of total cellular RNA using guanidine isothiocyanate (described, e.g., in U.S. Pat. No. 4,843,155) used in conjunction with, for example, oligo-dT strepavidin beads, is an example of one means of isolating mRNA. The RNA, as desirable, can be converted to cDNA using reverse transcriptase.

A wide range of techniques for isolating genomic DNA exist that are amenable for use in a variety of embodiments of the present invention. In some embodiments, only a portion of the total genomic DNA is isolated (e.g., on the basis of the chemical and/or physical state in which it is present (e.g., in a collection of cells)). For example, transcriptionally active and/or potentially active genes can be isolated (e.g., away from inactive sequences (e.g., using Dnase I digestion).

The present invention is not limited by the type of nucleic acid utilized (e.g., as a transfection molecule (e.g., that is introduced into cells for expression)) in a 2-D and/or 3-D transfection array described herein. In some embodiments, libraries of nucleic acid molecules are utilized in a transfection array. In some embodiments, an array may comprise a library of related, mutated sequences, such as a library of mutants of a particular protein, or libraries of potential promoter sequences, etc. A variety of forms of mutagenesis exist that are well known in the art that can be utilized to generate a combinatorial library. For example, homologs of a protein of interest can be generated and isolated from a library by screening using, for example, alanine scanning mutagenesis and the like (See, e.g., Ruf et al. (1994) Biochemistry 33:15 65-1572; Wang et al. (1994) J. Biol. Chem. 269:3095-3099; Balint et al. (1993) Gene 137:109-118; Grodberg et al. (1993) Eur. J. Biochem. 218:597-601; Nagashima et al. (1993) J. Biol. Chem. 268:2888-2892; Lowinan et al. (1991) Biochemistry 30:10832-10838; and Cunningham et al. (1989) Science 244:1081-1085), by linker scanning mutagenesis (See, e.g., Gustin et al. (1993) Virology 193:653-660; Brown et al. (1992) Mol. Cell. Biol. 12:2644-2652; McKnight et al. (1982) Science 232:316); by saturation mutagenesis (See, e.g., Meyers et al. (1986) Science 232:613); by PCR mutagenesis (See, e.g., Leung et al. (1989) Method Cell Mol Biol 1:11-19); and/or by random mutagenesis (See, e.g., Miller et al. (1992) A Short Course in Bacterial Genetics, CSHL Press, Cold Spring Harbor, N.Y.; and Greener et al. (1994) Strategies in Mol. Biol. 7:32-34).

In some embodiments, a transfection array (e.g., 2-D and/or 3-D array) of the present invention utilizes a library of small gene fragments (e.g., sequences that encode dominant-acting synthetic genetic elements (SGEs) (e.g., molecules that interfere with the function of genes from which they are derived (antagonists) or that are dominant constitutively active fragments (agonists) of such genes)). SGEs that can be identified by the subject method include, but are not limited to, polypeptides, inhibitory antisense RNA molecules (e.g., siRNAs, shRNAs, snRNAs, etc.), ribozymes, nucleic acid decoys, and small peptides.

In some embodiments, an SGE is identified using the compositions and methods of the present invention. These genetic elements might function to inhibit the function of an endogenous gene at the level of nucleic acid expression and/or translation (e.g., by an antisense or decoy mechanism. Alternatively, the synthetic genetic elements might encode a polypeptide that is inhibitory through a mechanism of interference at the protein level (e.g., a dominant negative fragment of the native protein). In some embodiments, a synthetic genetic element may function to enhance the function of an endogenous gene (e.g., by encoding a polypeptide that retains at least a portion of the bioactivity of the corresponding endogenous gene, and may in particular instances be constitutively active).

In some embodiments, a synthetic genetic element library is generated from total cDNA, that may be further fragmented, and provided in the form of an expression library. In some embodiments, the inserts in the library range from about 100 base pairs (bp) to about 700 bp, In some embodiments, from about 20 bp to about 100 bp, and in some embodiments, from about 30 bp to about 70 bp, although shorter and longer sequences are contemplated.

For cDNA-derived libraries, the nucleic acid library can be a normalized library containing roughly equal numbers of clones corresponding to each gene expressed in the cell type from which it was made, without regard for the level of expression of any gene.

Libraries can be generated to include both sense and antisense coding (and non-coding sequences) sequences. In some embodiments, expression (e.g., transcription) of library sequences in target/host cells creates antisense nucleic acids (e.g., RNAs) that inhibit transcription of the corresponding endogenous gene. In some embodiments, compositions and methods described in U.S. Pat. No. 5,702,898 (hereby incorporated by reference in its entirety) for normalizing a cDNA library constructed in a vector are utilized. In some embodiments, a sequence library is a subtractive cDNA library. Many strategies have been used to create subtractive libraries, and can be readily adapted for use in the present method. For example, use of directionally cloned cDNA libraries as starting material may be used (See, e.g., Palazzolo and Meyerowitz, (1987) Gene 52:197; Palazzolo et al. (1989) Neuron 3:527; Palazzolo et al. (1990) Gene 88:25). In some embodiments, a library of sequences is utilized that has been generated by providing an endonuclease restriction site at the 5′ end of an oligo(dT) primer. The restriction enzyme recognition sequence does not affect the annealing of the 12-20 base oligo(dT) primer to the mRNA, so the cDNA second strand synthesized from the first strand template includes the new recognition site added to the original 3′ end of the coding sequence. After second strand cDNA synthesis, a blunt ended linker molecule containing a second restriction site (or a partially double stranded linker adapter containing a protruding end compatible with a second restriction site) is ligated to both ends of the cDNA. The site encoded by the linker is now on both ends of the cDNA molecule, but only the 3′ end of the cDNA has the site introduced by the modified primer. Following the linker ligation step, the product is digested with both restriction enzymes (or, if a partially double stranded linker adapter was ligated onto the cDNA, with only the enzyme that recognizes the modified primer sequence). A population of cDNA molecules results which all have one defined sequence on their 5′ end and a different defined sequence on their 3′ end. In some embodiments, a sequence library is generated according to a directional cloning strategy developed by Meissner et al. (1987) PNAS 84:4171) that requires no sequence-specific modified primer, but rather a double stranded palindromic BamHI/HindIII directional linker having the sequence d(GCTTGGATCCAAGC), that is ligated to a population of oligo(dT)-primed cDNAs, followed by digestion of the ligation products with BamHI and HindIII. The palindromic linker, when annealed to double stranded form, includes an internal BamHI site (GGATCC) flanked by 4 of the 6 bases that define a HindIII site (AAGCTT). The missing bases needed to complete a HindIII site are d(AA) on the 5′ end or d(TT) on the 3′ end. Regardless of the sequence to which this directional linker ligates, the internal BamHI site will be present. However, HindIII can only cut the linker if it ligates next to an d(AA):d(TT) dinucleotide base pair. In an oligo(dT)-primed strategy, a HindIII site is always generated at the 3′ end of the cDNA after ligation to this directional linker. For cDNAs having the sequence d(TT) at their 5′ ends (statistically 1 in 16 molecules), linker addition will also yield a HindIII site at the 5′ end. However, because the 5′ ends of cDNA are heterogeneous due to the lack of processivity of reverse transcriptases, cDNA products from every gene segment will be represented in the library.

In some embodiments, a sequence library encodes molecules (e.g., nucleic acid sequences) that correspond to regulatory elements of a gene and that can inhibit expression of the gene by sequestering molecules important for transcription (e.g., transcriptional factors) thereby competing for the necessary components to express the endogenous gene.

In some embodiments, a sequence library can be generated by randomly fragmenting a single gene to obtain a random fragment expression library derived exclusively from the gene of interest. As a practical matter, such a library contains a much greater variety of sequences from the gene of interest than will a random fragment library prepared from total cDNA.

In some embodiments, a purified DNA corresponding to a gene or genome to be suppressed (e.g., within cells transfected in an array of the present invention) is first randomly fragmented by enzymatic, chemical, or physical procedures. In some embodiments, random fragments of DNA are produced by treating the DNA with a nuclease, such as DNase I. In some embodiments, the random DNA fragments can be incorporated as inserts in a sequence library. DNase I partial digestion and library construction are described generally in Molecular Cloning, A Laboratory Manual, Sambrook et al., Eds., Cold Spring Harbor Laboratory, Cold Spring Harbor, N.Y. (1989). In some embodiments, sequence fragment are expressed as part of a fusion protein. In some embodiments, the inserted sequence fragment alone may be expressed. In another embodiment, ribozyme-encoding sequences may be inserted directly adjacent to the insert to allow for selection of most efficient ribozyme-antisense clones. In still other embodiments the sequence library (e.g., gene suppression element library) may be further modified by random mutagenesis procedures known in the art. The inserted fragments may be expressed from either a constitutive or an inducible promoter.

In some embodiments, compositions and methods of the present invention utilize a library encoding a variegated population of small peptides (e.g., 4-25 amino acid residues in length). The library can be generated from coding sequences of total cDNA, or single genes, or can be random or semi-random in sequence.

In some embodiments, compositions and methods of the present invention utilize a transfection array that (e.g., when a nucleic acid sequence is transcribed in the host cell) gives rise to double stranded RNA (e.g, for use in identifying dsRNA constructs that produce a particular phenotype by RNA interference).

Libraries of coding sequences, whether encoding random peptides or full length proteins, may be expressed in many ways, including as portions of chimeric (e.g., fusion) proteins. In some instances it may be necessary to introduce an unstructured polypeptide linker region between portions of a chimeric protein derived from different proteins. The linker may facilitate enhanced flexibility of the chimeric protein allowing each portion to fold correctly and retain appropriate biological activity in the host cell. The linker can be of natural origin, such as a sequence determined to exist in random coil between two domains of a protein. Alternatively, the linker can be of synthetic origin. In some embodiments, naturally occurring unstructured linkers of human origin are preferred (e.g., because they reduce the risk of immunogenicity). In some embodiments, a fusion protein may comprise a secretion signal sequence (e.g., where secretion of a protein is desired).

The present invention is not limited by the number of different transfection molecules presented in any particular array (e.g., in any particular transfection zone/spot or on any give surface of an array).

In some embodiments, a transfection cell array provides, in a single array, at least 10 different sequences, more preferably at least 100, 1000 or even 10,000 different, discrete types of transfection molecules (e.g., nucleic acid sequences). In some embodiments, more than 10,000 or less than 10 different sequences are provided. In some embodiments, transfection molecules are arrayed in an addressable fashion, such as rows and columns (e.g., where the substrate is a planar surface).

The present invention contemplates the formation of arrays with a plurality of transfection zones/spots on a substrate surface (e.g., for both 2-D and 3-D arrays). In some embodiments, a substrate surface has about 10,000 transfection zones/spots in a one centimeter square area. In some embodiments, a transfection array provides a density of at least 1000 different transfection zones/spots per square centimeter, and more preferably at least 5000, 8000, 10000, 100000, or more transfection zones/spots. In some embodiments, lower or higher densities of transfection zones/spots are utilized.

As described herein, the present invention provides the presentation of two or more different transfection molecules at any given spot/transfection zone described herein (e.g. for co-transfection and/or co-expression of the two or more molecules in the same cells). Co-transfections can be performed with transfected cell microarrays if the solution spotted on the surface where reverse transfection occurs (e.g., in a 2-D array), or if the crosslinkable biopolymer and cells are mixed and (e.g., in a 3-D array) contains more than one plasmid or nucleic acid construct. The present invention contemplates the transfection of 2 or more, 10 or more, 50 or more, 100 or more transfection molecules within any given transfection zone.

In some embodiments, the ability to transfect two or more transfection molecules into cells in a cell array of the present invention provides a variety of uses. These include, but are not limited to, the ability to infer the expression of a gene product by detecting the expression of a co-transfected plasmid encoding a marker protein (e.g. GFP, luciferase, beta-galactosidase, or any protein to which a specific antibody is available), express all the components of a multi-subunit complex (e.g. the T-cell receptor) in the same cells, express all the components of a signal transduction pathway (e.g. MAP kinase pathway) in the same cells, and express all the components of a pathway that synthesizes a small molecule (e.g. intracellular antibodies, etc). In addition, the capacity to co-transfect allows the creation of microarrays with combinatorial combinations of co-expressed plasmids. This capacity is particularly useful for implementing mammalian two-hybrid assays in which plasmids encoding bait and prey proteins are co-transfected into the same cells by spotting them in one feature of the microarray.

The capacity to co-transfect is also useful when the goal is to promote differentiation of the transfected cells along a certain tissue lineage. For example, combination of genes can be expressed in a stem cell or early progenitor cell that may alter the differentiation of the cells (e.g., into endothelial, liver, heart, pancreatic, lymphoid, islet, brain, lung, kidney or other cell types). In this fashion, arrays can be made with primary-like cells that can be used to examine interactions of protein or small molecules that are cell-type specific.

Furthermore, transfection molecules may be introduced in specific locations on or within a 2-D or 3-D array, respectively. For example, combinations of cDNAs can be printed in different patterns on a substrate surface or be made to be included in a plurality of spatially isolated crosslinked biopolymers comprising cells and transfection molecules. Exemplary patterns include, but are not limited to, bulls-eyes, squares, rectangles of varying heights and widths, and lines of single cell thickness. By placing (e.g., in particular patterns) combinations of cDNAs that cause differentiation of cells into different tissue types, this technology can be used to obtain arrays with distinct cell types in distinct locations. This capacity can be useful when trying to create tissue-like structures on the array, such as blood capillaries and/or stromal structures, or when studying the response of one cell type to the protein secretions of another cell type. For example, a secreting cell type can be created in the center of a bulls-eye pattern and responder cell types of different tissues can be created on the edge of bulls-eye. The response of the responder cells to the secretions of the center cell can then be examined (e.g., utilizing compositions and methods described herein).

Arrays containing mixtures of plasmids at each transfection zone can be constructed by mixing plasmids before fixation to the substrate surface (e.g., printing, printing in serial, printing with masks, or printing with patterned printheads) or before mixing a crosslinking agent to a composition comprising a crosslinkable biopolymer, cells and transfection molecules. For example, plasmids could be mixed in a container (e.g., before printing and printed as a homogenous mixture).

A carrier and/or biopolymer useful in the compositions and methods of the present invention can be, for example, gelatin or an equivalent thereof. In certain embodiments, the carrier is a hydrogel, such as polycarboxylic acid, cellulosic polymer, polyvinylpyrrolidone, maleic anhydride polymer, polyamide, polyvinyl alcohol, or polyethylene oxide.

The present invention is not limited by the type of substrate surface to which transfection molecules are attached (e.g., for 2-D transfection arrays) or to which 3-D components (e.g., a crosslinkable biopolymer comprising transfection molecules and cells) are attached.

Any suitable surface that can be used to affix mixtures of the present invention to its surface can be used. For example, the surface can be glass, plastics (such as polytetrafluoroethylene, polyvinylidenedifluoride, polystyrene, polycarbonate, polypropylene), silicon, metal, (such as gold), membranes (such as nitrocellulose, methylcellulose, PTFE or cellulose), paper, biomaterials (such as protein, gelatin, agar), tissues (such as skin, endothelial tissue, bone, cartilage), minerals (such as hydroxylapatite, graphite) and/or a combination of the same. Additional compounds may be added to the base material of the surface to provide functionality. For example, scintillants can be added to a polystyrene substrate to allow Scintillation Proximity Assays to be performed. The substrate may be a porous solid support or non-porous solid support. The surface can have concave or convex regions, patterns of hydrophobic or hydrophilic regions, diffraction gratings, channels or other features. The scale of these features can range from the meter to the nanometer scale. For example, the scale can be on the micron scale for microfluidics channels or other MEMS features or on the nanometer scale for nanotubes or buckyballs. The surface can be planar, planar with raised or sunken features, spherical (e.g. optically encoded beads), fibers (e.g. fiber optic bundles), tubular (both interior or exterior), a 3-dimensional network (such as interlinking rods, tubes, spheres) or other shapes. The surface can be part of an integrated system. For example, the surface can be the bottom of a microtitre dish, a culture dish, and/or a culture chamber. Other components, such as lenses, gratings, and electrodes, can be integrated with the surface. In general, the material of the substrate and geometry of the array will be selected based on criteria that it be useful for automation of array formation, culturing and/or detection of cellular phenotype.

In still other embodiments, the solid support is a microsphere (bead), especially a FACS sortable bead. In some embodiments, each bead is an individual transfection zone/spot (e.g., having a homogenous population of target sequences and distinct from most other beads in the mixture, and one or more tags that can be used to the identify any given bead and therefore the sequence it displays). The identity of any given sequence that can induce a FACS-detectable change in cells that adhere to the beads can be readily determined from the tag(s) associate with the bead. For example, the tag can be an electrophoric tagging molecules that are used as a binary code (See, e.g., Ohlmeyer et al. (1993) PNAS 90:10922-10926). Exemplary tags are haloaromatic alkyl ethers that are detectable as their trimethylsilyl ethers at less than femtomolar levels by electron capture gas chromatography (ECGC). Variations in the length of the alkyl chain, as well as the nature and position of the aromatic halide substituents, may also be utilized. In some embodiments, a sequence is attached to a photocleavable linker and the tag is attached through a catechol ether linker via carbene insertion into the bead matrix (See, e.g., Nestler et al. (1994) J. Org. Chem. 59:4723-4724). This orthogonal attachment strategy permits the FACS sorting of the cell/bead entities and subsequent decoding by ECGC after oxidative detachment of the tag sets from isolated beads. In other embodiments, the beads can be tagged with two or more fluorescently active molecules, and the identity of the bead is defined by the ratio of the various fluorophores.

In some embodiments, the transfection array can be disposed on the end of a fiber optic system, such as a fiber optic bundle. Each fiber optic bundle contains thousands to millions of individual fibers depending on the diameter of the bundle. Changes in the phenotype of cells applied to the transfection array can be detected spectrometrically by conductance or transmittance of light over the spatially defined optic bundle. An optical fiber is a clad plastic or glass tube wherein the cladding is of a lower index of refraction than the core of the tube. When a plurality of such tubes are combined, a fiber optic bundle is produced. The choice of materials for the fiber optic will depend at least in part on the wavelengths at which the spectrometric analysis of the transfected cells is to be accomplished.

In some embodiments, a substrate surface can be coated with, for example, a cationic moiety. The cationic moiety can be any positively charged species capable of electrostatically binding to negatively charged polynucleotides. Preferred cationic moieties for use in the carrier are polycations, such as polylysine (e.g., poly-L-lysine), polyarginine, polyomithine, spermine, basic proteins such as histones (See, e.g., Chen et al. (1994) FEBS Letters 338:167-169), avidin, protamines (see e.g., Wagner et al. (1990) PNAS 87: 3410-3414), modified albumin (i.e., N-acylurea albumin) (See e.g., Huckett et al. (1990) Chemical Pharmacology 40: 253-263), and polyamidoamine cascade polymers (See e.g., Haensler et al. (1993) Bioconjugate Chem. 4:372-379). A preferred polycation is polylysine (e.g., ranging from 3,800 to 60,000 daltons). Alternatively, the surface itself can be positively charged (e.g., gamma amino propyl silane or other alkyl silanes).

The substrate surface can also be coated with molecules for additional functions. For example, molecules can be capture reagents such as antibodies, biotin, avidin, Ni-NTA to bind epitopes, avidin, biotinylted molecules, or 6-His tagged molecules. Alternatively, the molecules can be culture reagents such as extracellular matrix, fetal calf serum, collagen, etc.

In some embodiments, once a microarrays of transfected cells have formed (e.g., cDNAs in transfection zones/spots have entered cells and the cells have expressed the encoded gene products), the microarrays can be transferred onto a variety of surfaces. Surfaces can be flexible or non-flexible and porous or non-porous. The surfaces can be flat or patterned with concave or convex regions, patterns of hydrophobic or hydrophilic regions, diffraction gratings, channels or other features. The scale of these features can range from the meter to the nanometer scale. Examples of surfaces include, but are not limited to, glass, plastics (such as polytetrafluoroethylene, polyvinylidenedifluoride, polystyrene, polycarbonate, polypropylene), silicon, metal, (such as gold), membranes (such as nitrocellulose, methylcellulose, PTFE or cellulose, polyvinylidene fluoride (PVDF)), paper, biomaterials (such as protein, gelatin, agar), tissues (such as skin, endothelial tissue, bone, cartilage), minerals (such as hydroxylapatite, graphite). Furthermore, many of these surfaces can be derivatized to provide additional functionalities. For example, scintillants can be added to a polystyrene substrate to allow Scintillation Proximity Assays to be performed. In another example, nitrocellulose membranes can be covalently modified with metal chelators that immobilize metals, such as nickel or cobalt, and allow the selective binding of proteins carrying a specific amino acid sequence, such as a hexa-histidine tag.

In some embodiments, transfers are performed such that the entire cellular material on the microarray is transferred (e.g., both the endogenous and recombinant materials made by the cells (e.g., RNA or protein) are transferred); or such that only the recombinant material is transferred. In some embodiments, the transfer of the microarray to another surface is accomplished by directly contacting the microarray to the other surface and allowing the material to move to the new surface under the influence of a force, such as capillary forces (e.g., commonly referred to as “blotting”), electric or magnetic fields, vacuum suction forces, or other forces. The material may bind to the/new surface through an interaction mediated by hydrophobic, hydrophillic, Van der Waals, ionic or other forces, or through specific receptor-ligand interactions (e.g. antibody-epitope interactions) or by becoming entangled in the molecular structure of the other surface.

The ability to transfer cellular material from the microarrays to another surface has many potential uses. These include, but are not limited to, the capacity to detect cellular phenotypes or protein properties using techniques normally performed on specific surfaces and the capacity to in parallel purify the recombinant gene products expressed in the microarray. Examples of techniques normally performed on specific surfaces include western blotting, far-western blotting, southwestern blotting, surface plasmon resonance (SPR), mass spectroscopy, and others. These techniques normally require the immobilization of native or denatured proteins on nitrocellulose, nylon, paper, polyvinylidene fluoride (PVDF), or gold or other metal surfaces or membranes. Southwestern blotting is used to detect the interaction of a nucleic acid (such as DNA or RNA) with a protein. After transfer to an appropriate membrane, microarrays of cells expressing a collection of DNA binding proteins, such as transcription factors, can be used to identify binding proteins for genomic DNA sequence elements.

The transfer of microarrays to other surfaces is also useful for the in parallel purification of the recombinant proteins expressed on the microarray. The binding of proteins or small molecules with the microarray can be detected with autoradiography, fluorescence, mass spectroscopy, immunofluorescence, or calorimetry.

The present invention is not limited by the type of cells utilized in a transfection cell array of the present invention. Indeed, a variety of cells may be utilized.

Examples of host cells for generating arrays of the present invention include prokaryotes, yeast, or higher eukaryotic cells, including plant and animal cells, especially mammalian cells. Prokaryotes include gram negative or gram positive organisms.

In some embodiments, compositions and methods of the present invention method utilize cells derived from higher eukaryotes (e.g., metazoans, mammalian cells, primate cells and/or human cells). Other species of mammalian cells that may be utilized include canine, feline, bovine, porcine, mouse and rat. For instance, such cells can be hematopoietic cells, neuronal cells, pancreatic cells, hepatic cells, chondrocytes, osteocytes, or myocytes. The cells can be fully differentiated cells or progenitor/stem cells. The cells can be derived from normal or diseased tissue, from differentiated or undifferentiated cells, from embryonic or adult tissue. The cells maybe dispersed in culture, or can be tissues samples containing multiple cells that retain some of the microarchitecture of the organ.

In some embodiments, a transfection array of the present invention is used to transfect a cell that can be co-cultured with a target cell. A biologically active protein secreted by the cells expressing nucleic acids (e.g., genes) from the transfection array can diffuse to neighboring target cells and induce a particular biological response (e.g., proliferation or differentiation, or activation of a signal transduction pathway (e.g., that can be detected by phenotypic criteria)). Likewise, antagonists of a given factor can be selected by the ability of the cell producing a functional antagonist to protect neighboring cells from the effect of exogenous factor added to the culture media. The host and target cells can be in direct contact, or separated (e.g., by a cell culture insert).

If yeast cells are used, the yeast may be of any species that are cultivable and in the transfection array can be maintained upon transfection. Suitable species include Kluyverei lactis, Schizosaccharomyces pombe, and Ustilaqo maydis; Saccharomyces cerevisiae is preferred. Other yeast that can be used in practicing the present invention are Neurospora crassa, Aspergillus niger, Aspergillus nidulans, Pichia pastoris, Candida tropicalis, and Hansenula polymorpha. The term “yeast,” as used herein, includes not only yeast in a strictly taxonomic sense (i.e., unicellular organisms), but also yeast-like multicellular fungi or filamentous fungi.

The choice of appropriate host cell can be influenced by the choice of detection signal. For instance, reporter constructs can provide a selectable or screenable trait upon gain-of-function or loss-of-function induced by a target nucleic acid. The reporter gene may be an unmodified gene already in the host cell pathway, or it may be a heterologous gene (e.g., a “reporter gene construct” described herein). In other embodiments, second messenger generation can be measured directly in a detection step, such as mobilization of intracellular calcium or phospholipid metabolism, in which case the host cell should have an appropriate starting phenotype for activation of such pathways.

In some embodiments, host cells can be engineered to express other recombinant genes. For instance, the host cells can be engineered with a reporter gene construct, and the ability of members of the transfection array to alter the level of expression of the reporter gene can be characterized. For example, a transfection array can be assessed for members which encode transcriptional activators or transcriptional repressors of the reporter gene, and may include native and non-native sequences. For example, a host cell can be transfected with reporter gene construct including a promoter sequence for which a protein which binds that sequence is sought.

In some embodiments, a host cell can be engineered so as to have a loss-of-function or gain-of-function phenotype, and the ability of members of the transfection array to alter the phenotype assessed.

In some embodiments, a host cell is engineered to express a recombinant cell surface receptor, and the transfection array might encode a variegated library of gene products or peptides, and the ability of one or more members of that library to induce or inhibit signal transduction by the receptor is assessed. For example, the transfection array can provide a library of secreted peptides, and the ability of a given peptide to induce signal transduction is detected by the conversion of the cell to an autocrine phenotype.

The present invention is not limited by the method of detecting cellular characteristics (e.g., nucleic acid and/or protein expression, cellular signaling, division, etc.) in a 2-D and/or 3-D transfection array described herein.

A variety of methods can be used to detect the consequence of uptake, and in many embodiments, expression (e.g., transcription) of sequences within cells (e.g., present in a 2-D and/or 3-D array of the present invention). In a general sense, assays described herein provide means for determining if the target sequence is able to confer a change in the phenotype of the cell relative to the same cell but which lacks the target sequence. Such changes can be detected on a gross cellular level, such as by changes in cell morphology (e.g., membrane ruffling, rate of mitosis, rate of cell death, mechanism of cell death, dye uptake, and the like). In other embodiments, changes to the cell's phenotype, if any, can be detected by more focused means, such as the detection of the level of a particular protein (e.g., a selectable or detectable marker), or level of mRNA or second messenger). Changes in the cell's phenotype can be determined by assaying reporter genes (beta-galactosidase, green fluorescent protein, beta-lactamase, luciferase, chloramphenicol acetyl transferase), assaying enzymes, using immunoassays, staining with dyes (e.g. DAPI, calcofluor), assaying electrical changes, characterizing changes in cell shape, examining changes in protein conformation, counting cell number, etc. Other changes of interest could be detected by methods such as chemical assays, light microscopy, scanning electron microscopy, transmission electron microscopy, atomic force microscopy, confocal microscopy, image reconstruction microscopy, scanners, autoradiography, light scattering, light absorbance, NMR, PET, patch clamping, calorimetry, mass spectrometry, surface plasmon resonance, and/or time resolved fluorescence. Data could be collected at single or multiple time points and analyzed by the appropriate software (See, e.g., U.S. Patent Application Publication No. 20030228601, hereby incorporated by reference in its entirety)).

In some embodiments, transcriptional activity within a cell is detected and/or measured by a transcription product (e.g., by detecting transcriptional activation (or repression) of an indicator gene(s)). Detection of the transcription product includes detecting the gene transcript, detecting the product directly (e.g., by immunoassay) or detecting an activity of the protein (e.g., such as an enzymatic activity or chromogenic/fluorogenic activity). The indicator gene may be an unmodified endogenous gene of the host cell, a modified endogenous gene, or a part of a completely heterologous construct (e.g., part of a reporter gene construct).

In some embodiments, the indicator gene is an unmodified endogenous gene. For example, compositions and methods of the present invention may detect the transcriptional level of one or more endogenous genes.

In some embodiments, a heterologous reporter gene construct can be used to provide the function of an indicator gene. Reporter gene constructs are prepared by operatively linking a reporter gene with at least one transcriptional regulatory element (See, e.g., Example 1)

The present invention is not limited by any particular reporter gene construct. Many reporter genes and transcriptional regulatory elements are known to those of skill in the art and others may be identified or synthesized by methods known to those of skill in the art. Examples of reporter genes include, but are not limited to CAT (chloramphenicol acetyl transferase) (See, e.g., Alton and Vapnek (1979), Nature 282:864-869) luciferase, and other enzyme detection systems, such as beta-galactosidase; firefly luciferase (See, e.g., deWet et al. (1987), Mol. Cell. Biol. 7:725-737); bacterial luciferase (See, e.g., Engebrecht and Silverman (1984), PNAS 1:4154-4158; Baldwin et al. (1984), Biochemistry 23:3-663-3667); alkaline phosphatase (See, e.g., Toh et al. (1989) Eur. J. Biochem. 182:231-238, Hall et al. (1983) J. Mol. Appl. Gen. 2:101), human placental secreted alkaline phosphatase (See, e.g., Cullen and Malim (1992) Methods in Enzymol. 216:362-368); beta-lactamase, and GST.

Transcriptional control elements for use in the reporter gene constructs, or for modifying the genomic locus of an indicator gene include, but are not limited to, promoters, enhancers, and repressor and activator binding sites. Suitable transcriptional regulatory elements may be derived from the transcriptional regulatory regions of genes whose expression is linked to the desired phenotype sought from the arrayed library.

In some embodiments, the reporter gene is a gene whose expression causes a phenotypic change which is screenable or selectable. If the change is selectable, the phenotypic change creates a difference in the growth or survival rate between cells which express the reporter gene and those which do not. If the change is screenable, the phenotype change creates a difference in some detectable characteristic of the cells, by which the cells which express the marker may be distinguished from those which do not. Selection is preferable to screening in that it can provide a means for amplifying from the cell culture those cells which express a test polypeptide which is a receptor effector.

Many different types of information are contemplated to be generated using the compositions and methods of the present invention. For example, binding partners for molecules such as drugs, hormones, interleukins, or secreted proteins can be identified by incubating the compounds of interest (test agents) with an array that overexpresses potential targets within each array feature or combinations of potential targets within each cell of an array feature. Binding can be detected by methods such as SPR, SPA, TRF, or autoradiography.

An array can also be used to identify targets of an organism's immune response to cancer, an infectious or autoimmune disease, exposure to chemicals, or environmental changes. In some embodiments, the present invention facilitates drug target discovery by permitting the identification of an endogenous gene wherein inhibition or activation of the gene may be of therapeutic value. In some embodiments, loss of function of genes can be characterized. For example, transcription arrays that give rise to dsRNA in the host cell can be used to assess the loss-of-function of a particular gene (e.g., via RNAi or gene silencing (See, e.g., Fire A (1999) Trends Genet. 15:358-363; Sharp P A (1999) Genes Dev 13:139-141; Hunter C. (1999) Curr. Biol. 9:R440-R442; Baulcombe D.C. (1999) Curr. Biol. 9:R599-R601; Vaucheret et al. (1998) Plant J. 16:651-659)). In some embodiments, an array of the present invention is utilized to characterize a gene of interest.

In some embodiments, an array of the present invention is utilized to characterize candidate agents (e.g., for therapeutics (e.g., proteins, small molecules, oligonucleotides, etc.). In some embodiments, arrays are used to characterize compound libraries and/or potential drug candidates.

The ability of certain viruses to infect cells or enter cells via receptor-mediated endocytosis, and to integrate into host cell genome and express viral genes stably and efficiently have made them attractive candidates for the transfer of nucleic acid sequences (e.g., transfection molecules) into mammalian cells. In some embodiments, vectors of the present invention are viral vectors (e.g., phage or andenovirus vectors).

Although some viruses that can accept foreign genetic material are limited in the number of nucleotides they can accommodate and in the range of cells they infect, these viruses have been demonstrated to successfully effect gene expression. However, adenoviruses do not integrate their genetic material into the host genome and therefore do not require host replication for gene expression, making them ideally suited for rapid, efficient, heterologous gene expression. Techniques for preparing replication-defective infective viruses are well known in the art.

Of course, in using viral delivery systems, one will desire to purify the virion sufficiently to render it essentially free of undesirable contaminants, such as defective interfering viral particles or endotoxins and other pyrogens such that it will not cause any untoward reactions in the cell, animal or individual receiving the vector construct. A preferred means of purifying the vector involves the use of buoyant density gradients, such as cesium chloride gradient centrifugation.

A particular method for delivery of the expression constructs involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a tissue or cell-specific construct that has been cloned therein.

The expression vector comprises a genetically engineered form of adenovirus. Knowledge of the genetic organization or adenovirus, a 36 kb, linear, double-stranded DNA virus, allows substitution of large pieces of adenoviral DNA with foreign sequences up to 7 kb (See Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification.

Adenovirus is particularly suitable for use as a gene transfer vector because of its mid-sized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP (located at 16.8 map units (m.u.)) is particularly efficient during the late phase of infection, and all the mRNA's issued from this promoter possess a 5′-tripartite leader (TPL) sequence which makes them preferred mRNA's for translation.

In a current system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins (EIA and EIB; Graham et al., 1977). Since the E3 region is dispensable from the adenovirus genome (Jones and Shenk, 1978), the current adenovirus vectors, with the help of 293 cells, carry foreign DNA in either the E1, the D3 or both regions (Graham and Prevec, 1991). Recently, adenoviral vectors comprising deletions in the E4 region have been described (U.S. Pat. No. 5,670,488, incorporated herein by reference).

In nature, adenovirus can package approximately 105% of the wild-type genome (Ghosh-Choudhury et al., 1987), providing capacity for about 2 extra kb of DNA. Combined with the approximately 5.5 kb of DNA that is replaceable in the E1 and E3 regions, the maximum capacity of the current adenovirus vector is under 7.5 kb, or about 15% of the total length of the vector. More than 80% of the adenovirus viral genome remains in the vector backbone.

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

Racher et al. (1995) disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

Other than the requirement that the adenovirus vector be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

As stated above, the typical adenovirus vector according to the present invention is replication defective and will not have an adenovirus E1 region. Thus, it will be most convenient to introduce the transforming construct at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors as described by Karlsson et al. (1986) or in the E4 region where a helper cell line or helper virus complements the E4 defect.

Adenovirus growth and manipulation is known to those of skill in the art, and exhibits broad host range in vitro and in vivo. This group of viruses can be obtained in high titers, e.g., 10.sup.9 to 10.sup.11 plaque-forming units per ml, and they are highly infective. The life cycle of adenovirus does not require integration into the host cell genome. The foreign genes delivered by adenovirus vectors are episomal and, therefore, have low genotoxicity to host cells.

Adenovirus vectors have been used in eukaryotic gene expression (Levrero et al., 1991; Gomez-Foix et al., 1992) and vaccine development (Grunhaus and Horwitz, 1992; Graham and Prevec, 1992). Recombinant adenovirus and adeno-associated virus (see below) can both infect and transduce non-dividing human primary cells.

Adeno-associated virus (AAV) is an attractive vector system for use in the cell transduction of the present invention as it has a high frequency of integration and it can infect nondividing cells, thus making it useful for delivery of genes into mammalian cells, for example, in tissue culture (Muzyczka, 1992) or in vivo. AAV has a broad host range for infectivity (Tratschin et al., 1984; Laughlin et al., 1986; Lebkowski et al., 1988; McLaughlin et al., 1988). Details concerning the generation and use of rAAV vectors are described in U.S. Pat. No. 5,139,941 and U.S. Pat. No. 4,797,368, each incorporated herein by reference.

Studies demonstrating the use of AAV in gene delivery include LaFace et al. (1988); Zhou et al. (1993); Flotte et al. (1993); and Walsh et al. (1994). Recombinant AAV vectors have been used successfully for in vitro and in vivo transduction of marker genes (Kaplitt et al., 1994; Lebkowski et al., 1988; Samulski et al., 1989; Yoder et al., 1994; Zhou et al., 1994; Hermonat and Muzyczka, 1984; Tratschin et al., 1985; McLaughlin et al., 1988) and genes involved in human diseases (Flotte et al., 1992; Luo et al., 1994; Ohi et al., 1990; Walsh et al., 1994; Wei et al., 1994). Recently, an AAV vector has been approved for phase I human trials for the treatment of cystic fibrosis.

AAV is a dependent parvovirus in that it requires coinfection with another virus (either adenovirus or a member of the herpes virus family) to undergo a productive infection in cultured cells (Muzyczka, 1992). In the absence of coinfection with helper virus, the wild type AAV genome integrates through its ends into human chromosome 19 where it resides in a latent state as a provirus (Kotin et al., 1990; Samulski et al., 1991). rAAV, however, is not restricted to chromosome 19 for integration unless the AAV Rep protein is also expressed (Shelling and Smith, 1994). When a cell carrying an AAV provirus is superinfected with a helper virus, the AAV genome is “rescued” from the chromosome or from a recombinant plasmid, and a normal productive infection is established (Samulski et al., 1989; McLaughlin et al., 1988; Kotin et al., 1990; Muzyczka, 1992).

Typically, recombinant AAV (rAAV) virus is made by cotransfecting a plasmid containing the gene of interest flanked by the two AAV terminal repeats (McLaughlin et al., 1988; Samulski et al., 1989; each incorporated herein by reference) and an expression plasmid containing the wild type AAV coding sequences without the terminal repeats, for example pIM45 (McCarty et al., 1991; incorporated herein by reference). The cells are also infected or transfected with adenovirus or plasmids carrying the adenovirus genes required for AAV helper function. rAAV virus stocks made in such fashion are contaminated with adenovirus which must be physically separated from the rAAV particles (for example, by cesium chloride density centrifugation). Alternatively, adenovirus vectors containing the AAV coding regions or cell lines containing the AAV coding regions and some or all of the adenovirus helper genes could be used (Yang et al., 1994; Clark et al., 1995). Cell lines carrying the rAAV DNA as an integrated provirus can also be used (Flotte et al., 1995).

Retroviruses have promise as gene delivery vectors due to their ability to integrate their genes into the host genome, transferring a large amount of foreign genetic material, infecting a broad spectrum of species and cell types and of being packaged in special cell-lines (Miller, 1992).

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Concern with the use of defective retrovirus vectors is the potential appearance of wild-type replication-competent virus in the packaging cells. This can result from recombination events in which the intact sequence from the recombinant virus inserts upstream from the gag, pol, env sequence integrated in the host cell genome. However, new packaging cell lines are now available that should greatly decrease the likelihood of recombination (Markowitz et al., 1988; Hersdorffer et al., 1990).

Gene delivery using second generation retroviral vectors has been reported. Kasahara et al. (1994) prepared an engineered variant of the Moloney murine leukemia virus, that normally infects only mouse cells, and modified an envelope protein so that the virus specifically bound to, and infected, human cells bearing the erythropoietin (EPO) receptor. This was achieved by inserting a portion of the EPO sequence into an envelope protein to create a chimeric protein with a new binding specificity.

Other viral vectors may be employed as expression constructs in the present invention. Vectors derived from viruses such as vaccinia virus (Ridgeway, 1988; Baichwal and Sugden, 1986; Coupar et al., 1988), sindbis virus, cytomegalovirus and herpes simplex virus may be employed. They offer several attractive features for various mammalian cells (Friedmann, 1989; Ridgeway, 1988; Baichwal and Sugden, 1986; Coupar et al., 1988; Horwich et al., 1990).

With the recent recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. Chang et al. recently introduced the chloramphenicol acetyltransferase (CAT) gene into duck hepatitis B virus genome in the place of the polymerase, surface, and pre-surface coding sequences. It was cotransfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

In certain further embodiments, the vector will be HSV. A factor that makes HSV an attractive vector is the size and organization of the genome. Because HSV is large, incorporation of multiple genes or expression cassettes is less problematic than in other smaller viral systems. In addition, the availability of different viral control sequences with varying performance (temporal, strength, etc.) makes it possible to control expression to a greater extent than in other systems. It also is an advantage that the virus has relatively few spliced messages, further easing genetic manipulations. HSV also is relatively easy to manipulate and can be grown to high titers. Thus, delivery is less of a problem, both in terms of volumes needed to attain sufficient MOI and in a lessened need for repeat dosings.

In still further embodiments of the present invention, the nucleic acids to be delivered are housed within an infective virus that has been engineered to express a specific binding ligand. The virus particle will thus bind specifically to the cognate receptors of the target cell and deliver the contents to the cell. A novel approach designed to allow specific targeting of retrovirus vectors was recently developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

In various embodiments of the invention, nucleic acid sequence encoding a fusion protein is delivered to a cell as an expression construct. In order to effect expression of a gene construct, the expression construct must be delivered into a cell. As described herein, one mechanism for delivery is via viral infection, where the expression construct is encapsidated in an infectious viral particle. However, several non-viral methods for the transfer of expression constructs into cells also are contemplated by the present invention. In one embodiment of the present invention, the expression construct may consist only of naked recombinant DNA or plasmids (e.g., vectors comprising nucleic acid sequences of the present invention). Transfer of the construct may be performed by any of the methods mentioned which physically or chemically permeabilize the cell membrane. Some of these techniques may be successfully adapted for in vivo or ex vivo use, as discussed below.

In a further embodiment of the invention, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is an expression construct complexed with Lipofectamine (Gibco BRL).

Liposome-mediated nucleic acid delivery and expression of foreign DNA in vitro has been very successful (Nicolau and Sene, 1982; Fraley et al., 1979; Nicolau et al., 1987). Wong et al. (1980) demonstrated the feasibility of liposome-mediated delivery and expression of foreign DNA in cultured chick embryo, HeLa and hepatoma cells.

In certain embodiments of the invention, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1. In other embodiments, the delivery vehicle may comprise a ligand and a liposome. Where a bacterial promoter is employed in the DNA construct, it also will be desirable to include within the liposome an appropriate bacterial polymerase.

Certain receptor-mediated gene targeting vehicles comprise a cell receptor-specific ligand and a DNA-binding agent. Others comprise a cell receptor-specific ligand to which the DNA construct to be delivered has been operatively attached. Several ligands have been used for receptor-mediated gene transfer (Wu and Wu, 1987; Wagner et al., 1990; Perales et al., 1994; Myers, EPO 0273085), which establishes the operability of the technique. In certain aspects of the present invention, the ligand will be chosen to correspond to a receptor specifically expressed on the EOE target cell population.

In other embodiments, the DNA delivery vehicle component of a cell-specific gene targeting vehicle may comprise a specific binding ligand in combination with a liposome. The nucleic acids to be delivered are housed within the liposome and the specific binding ligand is functionally incorporated into the liposome membrane. The liposome will thus specifically bind to the receptors of the target cell and deliver the contents to the cell. Such systems have been shown to be functional using systems in which, for example, epidermal growth factor (EGF) is used in the receptor-mediated delivery of a nucleic acid to cells that exhibit upregulation of the EGF receptor.

In still further embodiments, the DNA delivery vehicle component of the targeted delivery vehicles may be a liposome itself, which will preferably comprise one or more lipids or glycoproteins that direct cell-specific binding. For example, Nicolau et al. (1987) employed lactosyl-ceramide, a galactose-terminal asialganglioside, incorporated into liposomes and observed an increase in the uptake of the insulin gene by hepatocytes. It is contemplated that the tissue-specific transforming constructs of the present invention can be specifically delivered into the target cells in a similar manner.

Homologous recombination (Koller and Smithies, 1992) allows the precise modification of existing genes, overcomes the problems of positional effects and insertional inactivation, and allows the inactivation of specific genes, as well as the replacement of one gene for another. Methods for homologous recombination are described in U.S. Pat. No. 5,614,396, incorporated herein in its entirety by reference. Thus, in some embodiments, host cells utilized in an array of the present invention are generated via homologous recombination. Homologous recombination relies, like antisense, on the tendency of nucleic acids to base pair with complementary sequences. In this instance, the base pairing serves to facilitate the interaction of two separate nucleic acid molecules so that strand breakage and repair can take place. In other words, the “homologous” aspect of the method relies on sequence homology to bring two complementary sequences into close proximity, while the “recombination” aspect provides for one complementary sequence to replace the other by virtue of the breaking of certain bonds and the formation of others.

In some embodiments, a candidate agent can be added to one or more transfection zones (e.g., to alter expression and/or activity). As used herein, a “candidate agent” may be any agent that potentially inhibits or enhances transcription, translation or the like within a cell, including, but not limited to, a drug, a pharmaceutical, a small molecule, and an compound. For example, the candidate agent may be a protein or fragment thereof, a small molecule, a chemical, or even a nucleic acid molecule. Using lead compounds to help develop improved compounds is know as “rational drug design” and includes not only comparisons with know inhibitors and enhancers of protein folding/solubility, but predictions relating to the structure of target molecules. Thus, using compositions and methods of the present invention, it is possible to identify candidate agents that have the ability to alter cellular activities (e.g., growth, transcription, cellular signaling, etc.).

The goal of rational drug design is to produce structural analogs of biologically active polypeptides or target compounds. By creating such analogs, it is possible to fashion drugs, which are more active or stable than the natural molecules, which have different susceptibility to alteration or which may affect the function of various other molecules. In one approach, it is possible to generate a three-dimensional structure for a target molecule, or a fragment thereof. This could be accomplished by x-ray crystallography, computer modeling or by a combination of both approaches.

It also is possible to use antibodies to ascertain the structure of a candidate enhancer or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

On the other hand, one may simply acquire, from various commercial sources, small molecule libraries that are believed to meet the basic criteria for useful drugs in an effort to “brute force” the identification of useful candidate agents. Screening of such libraries, including combinatorially generated libraries (e.g., peptide libraries), is a rapid and efficient way to screen large number of related (and unrelated) agents for activity. Combinatorial approaches also lend themselves to rapid evolution of potential drugs by the creation of second, third and fourth generation compounds modeled of active, but otherwise undesirable compounds.

Candidate agents may include fragments or parts of naturally-occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the agents (e.g., pharmaceuticals) to be screened could also be derived or synthesized from chemical compositions or man-made compounds. Thus, it is understood that the candidate agent identified by the present invention may be any peptide, polypeptide, polynucleotide, small molecule inhibitors or any other chemicals or compounds (e.g., that may be designed through rational drug design starting from known inhibitors or enhancers).

Other potential agents include antisense molecules, ribozymes, and antibodies (including single chain antibodies), each of which would be specific for the target molecule. Such compounds are described in greater detail elsewhere in this document. For example, an antisense molecule that bound to a translational or transcriptional start site, or splice junctions, would be ideal candidate inhibitors.

In addition to the modulating agents (e.g., compounds) initially identified, other sterically similar compounds may be formulated to mimic the key portions of the structure of the modulators. Such agents (e.g., compounds), which may include peptidomimetics of peptide modulators, may be used in the same manner as the initial modulators.

The invention also provides a method for screening for mutations in a host cell, or in a target protein sequence (e.g., that alters transcription in a cell). For example, cells comprising a transcription molecule of the present invention can be treated with a mutagen, and those host cells that display an increase in growth (e.g., rate or abundance) identified. A “mutagen” is intended to include, but not be limited to chemical mutagens such as ethyl methane sulphonate, N-methyl-N′-nitroso-guanidine and nitrous acid as well as physical agents such as ionizing radiation.

In an alternative embodiment, mutations can be introduced into a polynucleotide sequence encoding a target protein. The altered polynucleotide is then tested to determine whether the solubility of the target protein is changed (e.g., as monitored by growth in a selective environment, e.g., in the presence of ampicillin). Such mutations include, but are not limited to, mutations induced by a mutagen; site directed mutations that alter specific amino acid residues such as mutation of cysteine residues to eliminate disulfide bonds; deletions that remove sets of specific amino acids such as deletion of a continuous stretch of hydrophobic amino acids; and fusions of the target protein to a second, particularly soluble protein. In each case, the solubility of the target protein is assessed by determining growth of the host cells in a selective environment.

Where employed, mutagenesis can be accomplished by a variety of standard, mutagenic procedures. Mutation can involve modification of the nucleotide sequence of a single gene, blocks of genes or whole chromosome. Changes in single genes may be the consequence of point mutations which involve the removal, addition or substitution of a single nucleotide base within a DNA sequence, or they may be the consequence of changes involving the insertion or deletion of large numbers of nucleotides.

Mutations can arise spontaneously as a result of events such as errors in the fidelity of DNA replication or the movement of transposable genetic elements (transposons) within the genome. They also are induced following exposure to chemical or physical mutagens. Such mutation-inducing agents include ionizing radiations, ultraviolet light and a diverse array of chemical such as alkylating agents and polycyclic aromatic hydrocarbons all of which are capable of interacting either directly or indirectly (generally following some metabolic biotransformations) with nucleic acids. The DNA lesions induced by such environmental agents may lead to modifications of base sequence when the affected DNA is replicated or repaired and thus to a mutation. Mutation also can be site-directed through the use of particular targeting methods.

### Random Mutagenesis.

i) Insertional Mutagenesis

Insertional mutagenesis is based on the inactivation of a gene via insertion of a known DNA fragment. Because it involves the insertion of some type of nucleic acid (e.g., DNA) fragment, the mutations generated are generally loss-of-function, rather than gain-of-function mutations. However, there are several examples of insertions generating gain-of-function mutations. Insertion mutagenesis has been very successful in bacteria and Drosophila.

Transposable genetic elements are DNA sequences that can move (transpose) from one place to another in the genome of a cell. The first transposable elements to be recognized were the Activator/Dissociation elements of Zea mays. Since then, they have been identified in a wide range of organisms, both prokaryotic and eukaryotic.

Transposable elements in the genome are characterized by being flanked by direct repeats of a short sequence of DNA that has been duplicated during transposition and is called a target site duplication. Virtually all transposable elements whatever their type, and mechanism of transposition, make such duplications at the site of their insertion. In some cases the number of bases duplicated is constant, in other cases it may vary with each transposition event. Most transposable elements have inverted repeat sequences at their termini. These terminal inverted repeats may be anything from a few bases to a few hundred bases long and in many cases they are known to be necessary for transposition.

Prokaryotic transposable elements have been most studied in E. coli and Gram negative bacteria, but also are present in Gram positive bacteria. They are generally termed insertion sequences if they are less than about 2 kB long, or transposons if they are longer. Bacteriophages such as mu and D108, which replicate by transposition, make up a third type of transposable element. elements of each type encode at least one polypeptide a transposase, required for their own transposition. Transposons often further include genes coding for function unrelated to transposition, for example, antibiotic resistance genes.

Transposons can be divided into two classes according to their structure. First, compound or composite transposons have copies of an insertion sequence element at each end, usually in an inverted orientation. These transposons require transposases encoded by one of their terminal IS elements. The second class of transposon have terminal repeats of about 30 base pairs and do not contain sequences from IS elements.

Transposition usually is either conservative or replicative, although in some cases it can be both. In replicative transposition, one copy of the transposing element remains at the donor site, and another is inserted at the target site. In conservative transposition, the transposing element is excised from one site and inserted at another.

Eukaryotic elements also can be classified according to their structure and mechanism of transportation. The primary distinction is between elements that transpose via an RNA intermediate, and elements that transpose directly from DNA to DNA.

Elements that transpose via an RNA intermediate often are referred to as retrotransposons, and their most characteristic feature is that they encode polypeptides that are believed to have reverse transcriptionase activity. There are two types of retrotransposon. Some resemble the integrated proviral DNA of a retrovirus in that they have long direct repeat sequences, long terminal repeats (LTRs), at each end. The similarity between these retrotransposons and proviruses extends to their coding capacity. They contain sequences related to the gag and pol genes of a retrovirus, suggesting that they transpose by a mechanism related to a retroviral life cycle. Retrotransposons of the second type have no terminal repeats. They also code for gag- and pol-like polypeptides and transpose by reverse transcription of RNA intermediates, but do so by a mechanism that differs from that or retrovirus-like elements. Transposition by reverse transcription is a replicative process and does not require excision of an element from a donor site.

Transposable elements are an important source of spontaneous mutations, and have influenced the ways in which genes and genomes have evolved. They can inactivate genes by inserting within them, and can cause gross chromosomal rearrangements either directly, through the activity of their transposases, or indirectly, as a result of recombination between copies of an element scattered around the genome. Transposable elements that excise often do so imprecisely and may produce alleles coding for altered gene products if the number of bases added or deleted is a multiple of three.

Transposable elements themselves may evolve in unusual ways. If they were inherited like other DNA sequences, then copies of an element in one species would be more like copies in closely related species than copies in more distant species. This is not always the case, suggesting that transposable elements are occasionally transmitted horizontally from one species to another.

ii) Chemical Mutagenesis.

Chemical mutagenesis offers certain advantages, such as the ability to find a full range of mutant alleles with degrees of phenotypic severity, and is facile and inexpensive to perform. The majority of chemical carcinogens produce mutations in DNA. Benzo(a)pyrene, N-acetoxy-2-acetyl aminofluorene and aflotoxin B1 cause GC to TA transversions in bacteria and mammalian cells. Benzo(a)pyrene also can produce base substitutions such as AT to TA. N-nitroso compounds produce GC to AT transitions. Alkylation of the O4 position of thymine induced by exposure to n-nitrosoureas results in TA to CG transitions.

A high correlation between mutagenicity and carcinogenity is the underlying assumption behind the Ames test (McCann et al., 1975) which speedily assays for mutants in a bacterial system, together with an added rat liver homogenate, which contains the microsomal cytochrome P450, to provide the metabolic activation of the mutagens where needed.

In vertebrates, several carcinogens have been found to produce mutation in the ras proto-oncogene. N-nitroso-N-methyl urea induces mammary, prostate and other carcinomas in rats with the majority of the tumors showing a G to A transition at the second position in codon 12 of the Ha-ras oncogene. Benzo(a)pyrene-induced skin tumors contain A to T transformation in the second codon of the Ha-ras gene.

iii) Radiation Mutagenesis.

The integrity of biological molecules is degraded by the ionizing radiation. Adsorption of the incident energy leads to the formation of ions and free radicals, and breakage of some covalent bonds. Susceptibility to radiation damage appears quite variable between molecules, and between different crystalline forms of the same molecule. It depends on the total accumulated dose, and also on the dose rate (as once free radicals are present, the molecular damage they cause depends on their natural diffusion rate and thus upon real time). Damage is reduced and controlled by making the sample as cold as possible.

Ionizing radiation causes DNA damage and cell killing, generally proportional to the dose rate. Ionizing radiation has been postulated to induce multiple biological effects by direct interaction with DNA, or through the formation of free radical species leading to DNA damage. These effects include gene mutations, malignant transformation, and cell killing. Although ionizing radiation has been demonstrated to induce expression of certain DNA repair genes in some prokaryotic and lower eukaryotic cells, little is known about the effects of ionizing radiation on the regulation of mammalian gene expression (Borek, 1985). Several studies have described changes in the pattern of protein synthesis observed after irradiation of mammalian cells. For example, ionizing radiation treatment of human malignant melanoma cells is associated with induction of several unidentified proteins (Boothman et al., 1989). Synthesis of cyclin and co-regulated polypeptides is suppressed by ionizing radiation in rat REF52 cells, but not in oncogene-transformed REF52 cell lines (Lambert and Borek, 1988). Other studies have demonstrated that certain growth factors or cytokines may be involved in x-ray-induced DNA damage. In this regard, platelet-derived growth factor is released from endothelial cells after irradiation (Witte, et al., 1989).

In the present invention, the term “ionizing radiation” means radiation comprising particles or photons that have sufficient energy or can produce sufficient energy via nuclear interactions to produce ionization (gain or loss of electrons). An exemplary and preferred ionizing radiation is an γ-radiation. The amount of ionizing radiation needed in a given cell generally depends upon the nature of that cell. Typically, an effective expression-inducing dose is less than a dose of ionizing radiation that causes cell damage or death directly. Means for determining an effective amount of radiation are well known in the art.

In a certain embodiments, an effective expression inducing amount is from about 2 to about 30 Gray (Gy) administered at a rate of from about 0.5 to about 2 Gy/minute. Even more preferably, an effective expression inducing amount of ionizing radiation is from about 5 to about 15 Gy. In other embodiments, doses of 2-9 Gy are used in single doses. An effective dose of ionizing radiation may be from 10 to 100 Gy, with 15 to 75 Gy being preferred, and 20 to 50 Gy being more preferred.

Any suitable means for delivering radiation to a tissue may be employed in the present invention in addition to external means. For example, radiation may be delivered by first providing a radiolabeled antibody that immunoreacts with an antigen of the tumor, followed by delivering an effective amount of the radiolabeled antibody to the tumor. In addition, radioisotopes may be used to deliver ionizing radiation to a tissue or cell.

iv) In Vitro Scanning Mutagenesis.

Random mutagenesis also may be introduced (e.g., using error prone PCR, See Cadwell and Joyce, 1992). The rate of mutagenesis may be increased by performing PCR in multiple tubes with dilutions of templates.

One particularly useful mutagenesis technique is alanine scanning mutagenesis in which a number of residues are substituted individually with the amino acid alanine so that the effects of losing side-chain interactions can be determined, while minimizing the risk of large-scale perturbations in protein conformation.

In recent years, techniques for estimating the equilibrium constant for ligand binding using minuscule amounts of protein have been developed (See, e.g., U.S. Pat. Nos. 5,221,605 and 5,238,808, herein incorporated by reference in their entireties). The ability to perform functional assays with small amounts of material can be exploited to develop highly efficient, in vitro methodologies for the saturation mutagenesis of antibodies. The inventors bypassed cloning steps by combining PCR mutagenesis with coupled in vitro transcription/translation for the high throughput generation of protein mutants. Here, the PCR products are used directly as the template for the in vitro transcription/translation of the mutant single chain antibodies. Because of the high efficiency with which all 19 amino acid substitutions can be generated and analyzed in this way, it is now possible to perform saturation mutagenesis on numerous residues of interest, a process that can be described as in vitro scanning saturation mutagenesis (Burks et al., 1997).

In vitro scanning saturation mutagenesis provides a rapid method for obtaining a large amount of structure-function information including: (i) identification of residues that modulate ligand binding specificity, (ii) a better understanding of ligand binding based on the identification of those amino acids that retain activity and those that abolish activity at a given location, (iii) an evaluation of the overall plasticity of an active site or protein subdomain, (iv) identification of amino acid substitutions that result in increased binding.

v) Random Mutagenesis by Fragmentation and Reassmbly.

A method for generating libraries of displayed polypeptides is described in U.S. Pat. No. 5,380,721, herein incorporated by reference in its entirety. The method comprises obtaining polynucleotide library members, pooling and fragmenting the polynucleotides, and reforming fragments therefrom, performing PCR amplification, thereby homologously recombining the fragments to form a shuffled pool of recombined polynucleotides.

b. Site-Directed Mutagenesis

Structure-guided site-specific mutagenesis represents a powerful tool for the dissection and engineering of protein-ligand interactions. The technique provides for the preparation and testing of sequence variants by introducing one or more nucleotide sequence changes into a selected DNA.

Site-specific mutagenesis uses specific oligonucleotide sequences which encode the DNA sequence of the desired mutation, as well as a sufficient number of adjacent, unmodified nucleotides. In this way, a primer sequence is provided with sufficient size and complexity to form a stable duplex on both sides of the deletion junction being traversed. For example, a primer of about 17 to 25 nucleotides in length is preferred, with about 5 to 10 residues on both sides of the junction of the sequence being altered.

The technique typically employs a bacteriophage vector that exists in both a single-stranded and double-stranded form. Vectors useful in site-directed mutagenesis include vectors such as the M13 phage. These phage vectors are commercially available and their use is generally well known to those skilled in the art. Double-stranded plasmids are also routinely employed in site-directed mutagenesis, which eliminates the step of transferring the gene of interest from a phage to a plasmid.

In general, one first obtains a single-stranded vector, or melts two strands of a double-stranded vector, which includes within its sequence a DNA sequence encoding the desired protein or genetic element. An oligonucleotide primer bearing the desired mutated sequence, synthetically prepared, is then annealed with the single-stranded DNA preparation, taking into account the degree of mismatch when selecting hybridization conditions. The hybridized product is subjected to DNA polymerizing enzymes such as E. coli polymerase I (Klenow fragment) in order to complete the synthesis of the mutation-bearing strand. Thus, a heteroduplex is formed, wherein one strand encodes the original non-mutated sequence, and the second strand bears the desired mutation. This heteroduplex vector is then used to transform appropriate host cells, such as E. coli cells, and clones are selected that include recombinant vectors bearing the mutated sequence arrangement.

Comprehensive information on the functional significance and information content of a given residue of protein can best be obtained by saturation mutagenesis in which all 19 amino acid substitutions are examined. The shortcoming of this approach is that the logistics of multiresidue saturation mutagenesis are daunting (Warren et al., 1996, Zeng et al., 1996; Yelton et al., 1995; Hilton et al., 1996). Hundreds, and possibly even thousands, of site specific mutants must be studied. However, improved techniques make production and rapid screening of mutants much more straightforward. See, U.S. Pat. Nos. 5,798,208 and 5,830,650, herein incorporated by reference in their entireties, for a description of “walk-through” mutagenesis.

Other methods of site-directed mutagenesis are disclosed in U.S. Pat. Nos. 5,220,007; 5,284,760; 5,354,670; 5,366,878; 5,389,514; 5,635,377; and 5,789,166, herein incorporated by reference in their entireties.

In some embodiments, a variant (e.g., a mutant) includes “nonconservative” changes (e.g., replacement of a glycine with a tryptophan). Analogous minor variations can also include amino acid deletions or insertions, or both. Guidance in determining which amino acid residues can be substituted, inserted, or deleted without abolishing biological activity can be found using computer programs (e.g., LASERGENE software, DNASTAR Inc., Madison, Wis.).

vi. Directed Evolution.

In some embodiments, variants or other nucleic acid molecules of interest, may be produced by methods such as directed evolution or other techniques for producing combinatorial libraries of variants. The synthesis of degenerate oligonucleotides is well known in the art (See e.g., Narang, Tetrahedron Lett., 39:39 (1983); Itakura et al., Recombinant DNA, in Walton (ed.), Proceedings of the 3rd Cleveland Symposium on Macromolecules, Elsevier, Amsterdam, pp 273-289 (1981); Itakura et al., Annu. Rev. Biochem., 53:323 (1984); Itakura et al., Science 198:1056 (1984); Ike et al., Nucl. Acid Res., 11:477 (1983), herein incorporated by reference in their entireties). Such techniques have been employed in the directed evolution of proteins (See e.g., Scott et al., Science 249:386 (1980); Roberts et al., Proc. Natl. Acad. Sci. USA 89:2429 (1992); Devlin et al., Science 249: 404 (1990); Cwirla et al., Proc. Natl. Acad. Sci. USA 87: 6378 (1990); each of which is herein incorporated by reference; as well as U.S. Pat. Nos. 5,223,409, 5,198,346, and 5,096,815; each of which is incorporated herein by reference).

### EXPERIMENTAL

The following examples are provided in order to demonstrate and further illustrate certain preferred embodiments and aspects of the present invention and are not to be construed as limiting the scope thereof.

### Example 1

### Lithographic Methods and Materials for Generating Transfection Cell Arrays

Cells. Estrogen receptor (ER)-positive MCF-7/WS8 mammary carcinoma cells, clonally derived from MCF-7 cells by selection for sensitivity to growth simulation by 17-β-estradiol (E₂) (See, e.g., Jiang et al., 1992. Mol Cell Endocrinol 90(1):77-86′ Levenson and Jordan, 1997. Cancer Res 57(15):3071-8), were utilized. Cells were cultured in fully estrogenized, phenol red-containing RPMI-1640 media supplemented with 10% fetal bovine serum (FBS), 100 μM non-essential amino acids, 100 units antibiotic/antimycotic, 2 mM L-glutamine, and 6 ng/ml insulin and maintained at 37° C. in a humidified 5% CO₂ atmosphere. Prior to transfecting cells for an experiment, cells were cultured under estrogen-free conditions by substituting phenol red-free RPMI-1640 and dextran-coated charcoal-treated fetal bovine serum in the medium. For experiments in which transfected cells were assayed in 24-well plates using a luminometer, or imaged in arrays using a CCD camera, cells were cultured under estrogen-free condition for 4 days or 18 h, respectively, prior to seeding. Culture in estrogen-free media for either time period allowed adequate time for upregulation of ER protein levels due to E₂ withdrawal, while the shorter culture period enhanced cell viability in the array. All media and media components were purchased from GIBCO/INVITROGEN (Carlsbad, Calif.).

Plasmids. Plasmids were purified from bacteria culture using QIAGEN (Valencia, Calif.) reagents and stored in Tris-EDTA buffer solution (10 mM Tris, 1 mM EDTA, pH 7.4)) or water at −20° C. Plasmid pEGFP-LUC encodes both the enhanced green fluorescent protein (EGFP) and firefly luciferase protein, under the direction of a CMV promoter (CLONTECH, Mountain View, Calif.). Plasmid pLUC encodes the firefly luciferase gene in the pNGVL1 (National Gene Vector Labs, University of Michigan) vector backbone with a CMV promoter. Estrogen-responsive plasmid pERE(3×)TK-ffLUC (See, e.g., Catherino and Jordan, 1995. Cancer Lett 92(1):39-47) contains three tandem copies of the palindromic estrogen response element (ERE) sequence, placed upstream of a minimal herpes simplex thymidine kinase (TK) promoter, directing expression of the firefly luciferase coding sequence in response to transcriptional activation by estradiol (E₂)-bound ERα, followed by recruitment of cofactor complexes and basal transcriptional machinery. Plasmid pTK-rLUC (phRL-TK, PROMEGA, Madison, Wis.) contains the minimal TK promoter driving expression of a humanized renilla luciferase and was used for normalization of the firefly luciferase plasmids. Plasmid pβGAL encodes for nuclear-targeted β-galactosidase in the pNGVL1 (National Gene Vector Labs, University of Michigan) vector backbone with a CMV promoter and was used for control spots on the array.

DNA complex formation. DNA complexes were formed with LIPOFECTAMINE 2000 (INVITROGEN, Carlsbad, Calif.), LIPOFECTAMINE LTX (INVITROGEN, Carlsbad, Calif.) or EFFECTENE (QIAGEN, Valencia, Calif.), following manufacturer's instructions. Briefly, for both LIPOFECTAMINE 2000 and LIPOFECTAMINE LTX, DNA complexes were formed at a DNA:lipid ratio of 1:2 in serum-free, OPTI-MEM media (INVITROGEN, Carlsbad, Calif.), by adding transfection reagent diluted in media dropwise to DNA in media, mixing by gentle pipeting, and then incubating for 20 minutes. EFFECTENE complexes were formed by diluting DNA into EC buffer, to which the Enhancer buffer was added at a DNA to Enhancer ratio of 1:8. After 2-5 minutes of incubation at room temperature, the EFFECTENE transfection reagent was then added to the DNA/Enhancer mixture at a DNA to Transfection reagent ratio of 1:4. After incubation at room temperature for 10 minutes, complexes were diluted with serum-free media before addition to surfaces or cells. DNA in complexes containing multiple plasmids was extensively mixed prior to complex formation. For induction studies in estrogen-free media, phenol red-free OPTI-MEM media was used for complex formation.

Multiwell dish format reporter gene assays. Multiwell dish format reporter gene assays were performed (e.g., to compare the ability of surface delivery of complexes to monitor ERα response with traditional bolus delivery). For surface delivery, the surface of wells of a 24-well plate (BECTON DICKINSON, Franklin Lakes, N.J.) were serum-coated by incubation with dextran-coated charcoal-stripped FBS (10% in 1×PBS, pH 7.4, 380 μL) for 18 hours at 4° C., followed by two wash steps with PBS (See, e.g., Bengali et al., 2005 Biotechnol Bioeng 90(3):290-302). Complexes were then immobilized following complex formation, as described above, by incubation of DNA complexes (475 μL) with the serum-coated wells for 2 hours. After complex incubation, the wells were washed twice with OPTI-MEM (for LIPOFECTAMINE 2000 complexes) or EC buffer (for EFFECTENE complexes) and 250,000 MCF-7 cells (cultured in estrogen-free media for 4 days) were seeded onto the immobilized DNA-lipid complexes in each well.

For bolus delivery, MCF-7 cells cultured in estrogen-free media for 4 days were seeded in estrogen-free medium into 24-well plates at densities of 125,000 cells per well. Eighteen hours later, complexes formed as described above were diluted in antibiotic-free, estrogen-free media and then added to the cells.

For both surface and bolus delivery, complexes contained both the pERE(3×)TK-ffLUC plasmid and the normalization plasmid, pTK-rLUC, at a ratio of 4:1. Total DNA amounts added for surface delivery ranged from 0.13-1.32 μg/cm² (0.25-2.5 μg per well) and 0.05-0.26 μg/cm² (0.025-0.5 μg per well) for bolus delivery. Given binding profiles, these ranges result in approximately the same amount of DNA bound to the surface as delivered as a bolus (See, e.g., 2005, Biotechnol Bioeng 90(3):290-302).

Immediately after complex addition for bolus delivery and 4 hours after cell seeding for surface delivery, cells were treated with combinations of E₂ (SIGMA-ALDRICH, St. Louis, Mo.), the complete anti-estrogen fulvestrant (FUL), also termed ICI 182,780, TOCRIS Bioscience, Ellisville, Mo.) or vehicle controls. E₂ and FUL were both dissolved in ethanol and diluted in estrogen-free media to obtain the indicated concentrations (10⁻¹² to 10⁻⁹M for E₂; 10⁻⁶M for FUL) prior to addition to cells. Ethanol diluted in estrogen-free media served as the vehicle control. Cells were harvested and assayed for firefly and renilla luciferase reporter gene activities 48 hours after transfection using the Dual-Luciferase Reporter assay system (PROMEGA, Madison, Wis.). In this dual-luciferase system, firefly and renilla luciferases are measured sequentially, in a single well. These measurements are accomplished by adding the firefly luciferase substrate first, measuring luminescence, and then adding reagents that quench the firefly luciferase reaction and simultaneously provide the renilla luciferase substrate, followed by measuring renilla luciferase activity. The dual-luciferase assays were carried out using an automated microplate luminometer equipped with dual-injection ports (MITHRAS LB 940, Berthold Technologies, Oak Ridge, Tenn.). Relative dual-luciferase activity was calculated by dividing the luminescent signal from the firefly reporter gene by the renilla luminescent signal.

Array fabrication. Soft lithography techniques were used to pattern DNA complex deposition. A polydimethylsiloxane (PDMS) mold was fabricated by curing PDMS into thin, flat disks. Briefly, PDMS was prepared in a 10:1 (v:v) ratio of Silicone Elastomer-184 and Silicone Elastomer Curing Agent-184 (SYLGARD 184, DOW CORNING, Midland, Mich.) by mixing the base and curing agent at least 50 times using a syringe mixing system. After allowing all air bubbles to escape, the PDMS was poured directly into a polystyrene tissue culture dish (100 mm, Corning, Corning, N.Y.) and cured at 60° C. for approximately 2 hours. The cured PDMS was removed from the dish and rods of precise diameters were then used to punch holes into the PDMS, with diameters of 2.4 mm. The PDMS mold was rinsed in 70% ethanol, oxidized using oxygen plasma and then reversibly sealed to sterile polystyrene microscope slides (NUNC, Rochester, N.Y.), which were fitted into custom-fabricated TEFLON slide holders that are rinsed in 70% ethanol before use. The holes in the PDMS mold, termed microwells, served as reservoirs for deposition of DNA complexes onto the polystyrene slide. After 2 hours of complex deposition in humid conditions, the PDMS mold was peeled away from the polystyrene, and the slide was rinsed thoroughly with OPTI-MEM. For all array studies, DNA concentrations ranged from 0.007 μg/μL to 0.021 μg/μL, with 2.2 μL to 4 μL of complex volume added to the microwells of the PDMS mold.

To visualize DNA complex immobilization on the array and verify deposition replicated the pattern of the microwells in the PDMS mold, plasmid (pEGFP-LUC) was labeled with tetramethyl rhodamine (LABEL IT Nucleic Acid Labeling Kit, MIRUS, Madison, Wis.), complexed as described above, and deposited in the microwells. After deposition, PDMS removal and rinsing, the resulting spots were visualized with fluorescence microscopy.

Transfection of cells on the array was verified by depositing complexes formed with plasmid pEGFP-LUC in the microwells, and imaging with fluorescence microscopy. After complex deposition, PDMS removal and rinsing, MCF-7 cells were seeded onto the slide at a density of 10⁶ cells per slide (18.75 cm²). Transfection was analyzed after 24 and 48 hours and characterized through GFP expression. Transfected cells were visualized using an epifluorescence microscope (LEICA; Bannockburn, Ill.) with a FITC filter and equipped with a digital camera. Transfection, as assayed through bioluminescence imaging, was verified by depositing complexes containing both pLUC and pTK-rLUC plasmids at a 1:1 ratio. After deposition, PDMS removal and rinsing, cells were seeded as described above. Transfection was analyzed after 24 hours and characterized by dual-luciferase expression through light emission.

For induction studies in the array, complexes formed with different plasmids were immobilized in different spots of the array, in triplicate. Briefly, complexes were formed with pLUC, pERE(3×)TK-ffLUC, pERE(3×)TK-ffLUC and pTK-rLUC (2:1 ratio), or pβGAL. After deposition, PDMS removal and rinsing, MCF-7 cells that had been cultured in estrogen-free media for 18 hours were seeded in estrogen-free medium on arrays at a density of 10⁶ cells per slide. Immediately after cell seeding, cells were treated with combinations E₂, FUL, or vehicle control, as described above. Dual-luciferase levels were analyzed 24 hours later by bioluminescence imaging.

Bioluminescence imaging. Expression of both luciferase reporter genes was assessed through imaging of light production upon sequential addition of the luciferase substrates to the bulk media. Bioluminescence imaging of the array was performed using an IVIS imaging system (XENOGEN Corp., Alameda, Calif.), which utilizes a cooled CCD camera. For imaging, a modified renilla luciferase substrate, VIVIREN (Promega, Madison, Wis.), was diluted to 0.66 mM in serum-containing media and then added to the arrays at a final concentration of 10 μM. After 2 minutes, the arrays were placed into a light-tight chamber and bioluminescence images were acquired for a total exposure time of 1 minute. Immediately following imaging with VIVIREN, 1 mM of luciferase substrate D-luciferin (Molecular Therapeutics Inc., MI, 20 mg/mL in PBS), was added into the media above the cells cultured on the array and bioluminescence images acquired 3 minutes later, with 1 minute exposure. Gray scale and bioluminescence images were superimposed using the LIVING IMAGE software (XENOGEN Corp., Alameda, Calif.). A constant size region of interest (ROI) was drawn over the spots of the array to calculate light signals. The signal intensity was reported as an integrated light flux (photons/s), determined by IGOR software (WAVEMETRICS, Lake Oswego, Oreg.). The signal due to firefly luciferase was determined by subtracting VIVIREN signal from the luciferin signal. Normalization was accomplished by dividing the firefly luciferase signal (luciferin signal minus VIVIREN signal) by the renilla luciferase signal (VIVIREN signal). A renilla signal threshold was set at 3.5E4 photon/s (2× background) to distinguish spots of unreliable signals indicating insufficient transfection.

Statistics. Statistical analysis was performed using JMP software (SAS Institute, Inc., Cary, N.C.). Comparative analyses were completed using one-way ANOVA with Tukey post-tests, at a 95% confidence level. Mean values with standard deviation are reported.

### Example 2

### Multiwell Dish Format Estrogen Responsive Element (ERE)-Reporter Gene Induction Studies

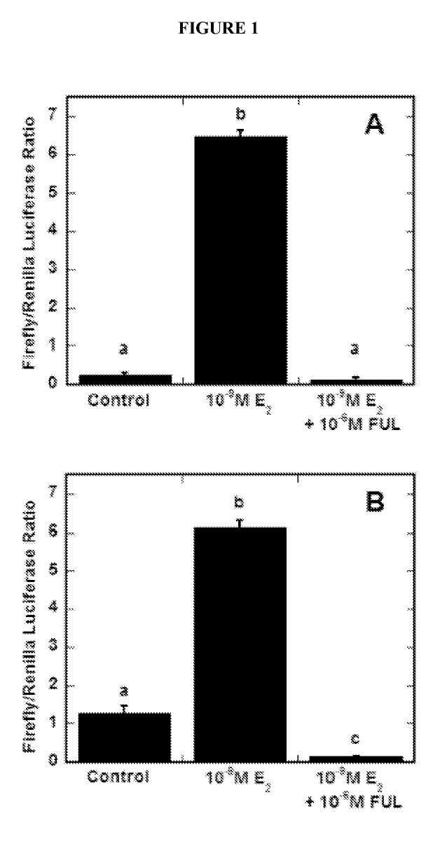

Multiwell dish format reporter gene assays were performed to compare estrogen receptor-alpha (Erα)-regulated, estrogen response element (ERE)-dependent transcriptional activity in MCF-7 cells transfected via surface-mediated delivery of DNA complexes in comparison to traditional bolus delivery (See FIG. 1). DNA complexes formed using an E₂-responsive firefly luciferase reporter plasmid pERE(3×)TK-ffLUC and a normalization plasmid pTK-rLUC encoding renilla luciferase were delivered to cells via bolus or surface delivery. Transfected cells were treated with various combinations of the agonist E₂, the complete antiestrogen FUL, or ethanol. Surface delivery of the plasmids (See FIG. 1B) resulted in E₂-stimulated responses similar to bolus delivery (See FIG. 1A), with E₂ statistically inducing firefly luciferase expression 6-7 fold (p<0.001) over vehicle control or the addition of FUL. Thus, the present invention provides that the physiologic state of the cells during surface-mediated delivery allowed the cells to transcriptionally respond to E₂. Further, the maximal induction of reporter gene activity was similar whether the DNA complexes were delivered via bolus or surface-mediated techniques.

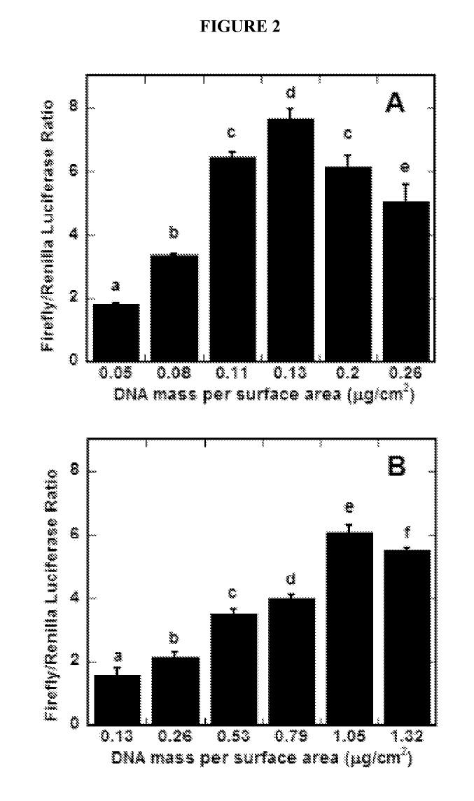

The amount of transfected plasmid was subsequently investigated, which indicated a similar DNA mass-dependent effect in reporter gene activity for both surface and bolus-mediated transfection methods (See FIG. 2). For bolus delivery (See FIG. 2A), all DNA amounts resulted in significantly different responses (p<0.01), except for 0.11 and 0.2 μg/cm², which were not statistically different from each other. Maximal induction was achieved at 0.13 μg/cm² (0.25 μg per well). For surface delivery (See FIG. 2B), all DNA amounts resulted in significantly different responses (p<0.05), with 1.05 μg/cm² (2 μg per well) corresponding to the highest induction by E₂. These results indicate that sufficient amounts of DNA must be transfected for optimal reporter gene activity and excess amounts of DNA lead to less efficient reporter gene activity, possibly due to toxicity, for both delivery methods.

Assuming that approximately 20% of DNA added to the cell culture dish surface is immobilized (See, e.g., Bengali et al., Biotechnol Bioeng 90(3):290-302) 2005), the condition with the highest induction (1.05 μg/cm²) would have presented approximately 0.21 μg/cm² of DNA to the cells, which is higher than the bolus condition with the highest induction (0.13 μg/cm²), but still in the range of robust activity. Therefore, surface delivery required more DNA added to the surface than what would have been expected given binding profiles (See, e.g., Bengali et al., Biotechnol Bioeng 90(3):290-302) 2005). Although an understanding of the mechanism is not necessary to practice the present invention and the present invention is not limited to any particular mechanism of action, in some embodiments, the requirement for more DNA may be due to lower than anticipated binding efficiencies (˜10%, but still within the range of profiles reported).

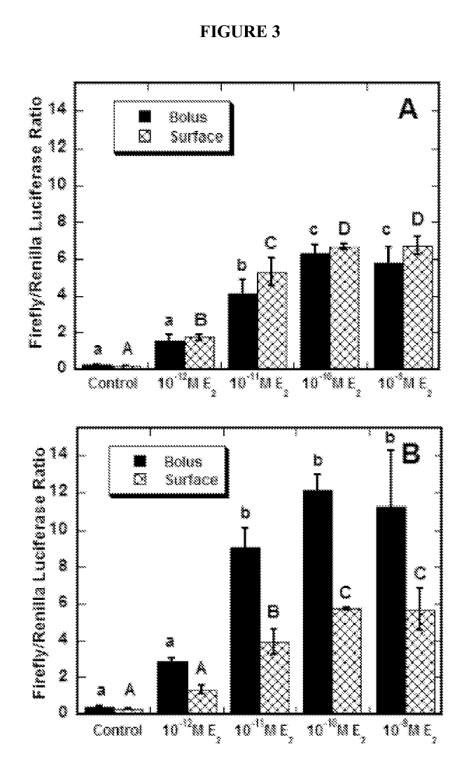

The specific transfection reagent used to form DNA complexes and E₂ concentration responses were subsequently investigated to determine the applicability and sensitivity of the reporter system (See FIG. 3). For LIPOFECTAMINE 2000-DNA complexes (See FIG. 3A), E₂-induction profiles were not significantly different using bolus versus surface delivery (See FIG. 3A), with E₂ eliciting a concentration response from 10⁻¹² M to 10⁻⁹ M (p<0.05), and maximal responsiveness observed from 10⁻¹² M to 10⁻⁹M E₂ (p>0.05) for both delivery methods. For EFFECTENE complexes (See FIG. 3B), bolus delivery resulted in statistically higher levels of ERE induction (p<0.05) than surface delivery for all concentrations of E₂, except control. However, the level of ERE induction for surface-mediated delivery was similar whether complexing DNA with EFFECTENE (See FIG. 3B) or with LIPOFECTAMINE 2000 (See FIG. 2A). Therefore, the particular transfection reagent used affected transcriptional activity via the conventional bolus delivery, but not via surface delivery. Thus, the present invention provides that either EFFECTENE or LIPOFECTAMINE 2000 can be used to delivery plasmid via surface-mediated transfection. Additionally, other transfection reagents could easily be adapted by one of ordinary skill in the art for use in surface-mediated delivery.

### Example 3

### Array Fabrication and Verification

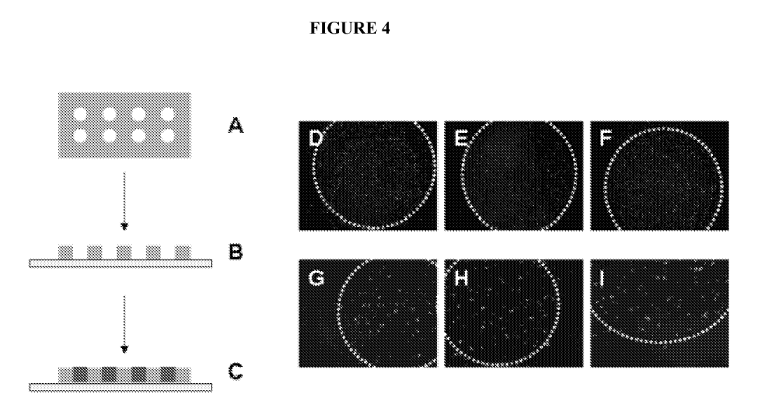

An array was created using soft lithography techniques to pattern DNA-lipid complex deposition and subsequent transfection upon cell seeding (See FIG. 4). Briefly, a polydimethylsiloxane (PDMS) mold with microwells (e.g., generated as described in Example 1) (See FIG. 4A) was reversibly sealed to polystyrene microscope slides (See FIG. 4B) with the microwells serving as reservoirs for deposition of DNA complexes onto the polystyrene slide (See FIG. 4C). Rhodamine-labeled DNA complexes deposited within microwells were immobilized to the slide in distinct regions, replicating the pattern of microwells in the PDMS mold (See FIG. 4D-F). Transfection of MCF-7 cells seeded onto arrays of complexes was determined by GFP expression, and was also confined to the patterns (See FIG. 4G-I).

### Example 4

### Bioluminescence Imaging of the Array

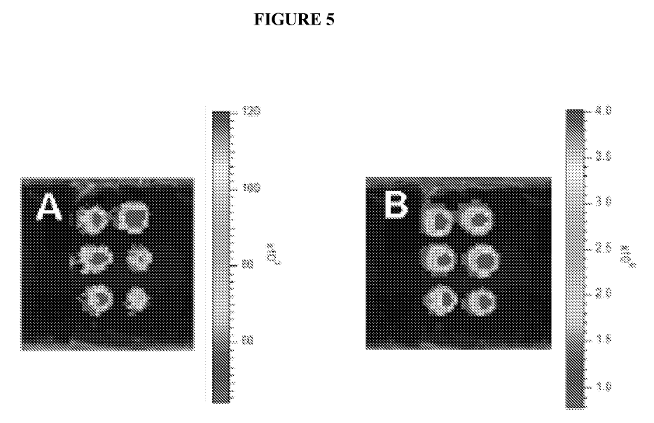

Arrays formed with complexes containing plasmids encoding firefly and renilla luciferase reporter genes (pLUC and pTK-rLUC) were used to verify the ability of bioluminescence imaging to detect dual-luciferase expression (See FIG. 5). Transfection of MCF-7 cells seeded onto these arrays was assayed after 24 hours by sequentially adding the renilla and firefly luciferase substrates. Following VIVIREN addition, spot intensities averaged 1.10×10⁵±2.56×10⁴ photon/s (See FIG. 5A), which are similar to signals obtained with arrays of only pTK-rLUC plasmid. D-Luciferin was subsequently added to the same array, which was then imaged to acquire a dual signal (See FIG. 5B), with average spot intensities of 3.66×10⁶±4.34×10⁵ photon/s. Firefly luciferase expression was determined by subtracting the initial VIVIREN signal from the signal obtained through imaging with D-luciferin. Firefly expression averaged 3.55×10⁶±4.30×10⁵ photon/s, also similar to intensities obtained with arrays formed with only rLUC plasmid. After normalization, the firefly luciferase signal was 34±8 fold greater than the respective renilla expression. Timecourse studies revealed that the VIVIREN signal remained constant for 10 minutes after substrate addition. Therefore, the present invention provides that the firefly luciferase signal can be obtained using a dual imaging strategy (e.g., as described herein) followed by subtraction techniques (e.g., since imaging can be accomplished within 10 minutes of VIVIREN addition. The present invention also provides that bioluminescence imaging is able to sensitively capture both luciferase signals, thereby enabling the same cell population to be analyzed for the expression of multiple reporter genes.

### Example 5

### Array Format ERE-Reporter Gene Induction Studies

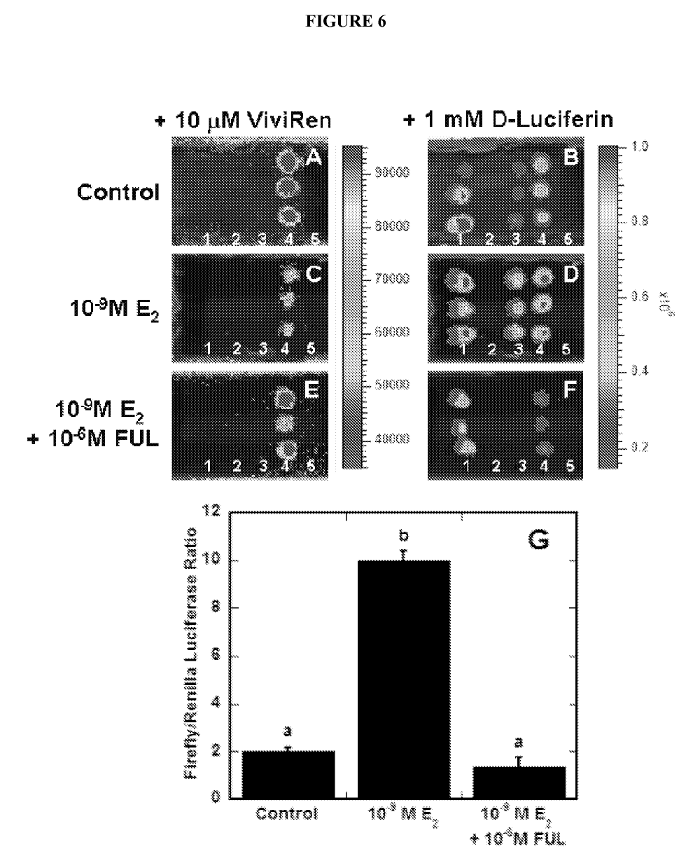

In order to assess the ability of the arrays to monitor induction of ERα transcriptional activity (See FIG. 6), complexes formed with different plasmids were immobilized as an array as follows: 1. pLUC; 2. no DNA (mock); 3. pERE(3×)TK-ffLUC; 4. pERE(3×)TK-ffLUC and pTK-rLUC (2:1 ratio); and 5. pβGAL. Cells seeded on the arrays were treated with combinations of ethanol control (See FIG. 6A-B), 10⁻⁹ M E₂ (See FIG. 6C-D), or 10⁻⁹ M E₂+10⁻⁶ M FUL (See FIG. 6E-F). Dual-luciferase levels were analyzed 24 hours later using bioluminescence imaging, by first imaging with VIVIREN (See FIG. 6A, C, E), and then imaging each array with D-luciferin (See FIG. 6B, E, F). Renilla luciferase activity was only detected in cells transfected with pTK-rLUC plasmid (See FIG. 6A, C, E, column 4), and not in cells transfected with only firefly luciferase-encoding plasmids (See FIG. 6A, C, E, columns 1 and 3), a control βGAL-encoding plasmid (See FIG. 6A, C, E, column 5) or no DNA (See FIG. 6A, C, E, column 2). Accordingly, firefly luciferase activity was only detected in cells transfected with pLUC (See FIG. 6B, D, F, column 1) or pERE(3×)TK-ffLUC (See FIG. 6B, D, F, columns 3 and 4), but not in mock or βGAL control transfected cells (See FIG. 6B, D, F, columns 2 and 5). These results verify the specificity of renilla and firefly luciferase detection in this system.

Firefly luciferase activity was detected at substantially higher levels in cells transfected with pERE(3×)TK-ffLUC and treated with E₂ (See FIG. 6D, columns 3 and 4) compared to those treated with ethanol (See FIG. 6B, columns 3 and 4) or E₂+FUL (See FIG. 6 F, columns 3 and 4). In control-treated arrays, spots of highest intensity were visualized for pLUC (See FIG. 6B, column 1), given its highly active CMV promoter. Cells transfected with both the pERE(3×)TK-ffLUC and pTK-rLUC plasmids (See FIG. 6B, column 4) resulted in higher signal intensities in the presence of luciferin than cells transfected with only the pERE(3×)TK-ffLUC (See FIG. 6B, column 3), as there was no carryover of VIVIREN signal in the latter spots of transfected cells without pTK-rLUC. For E₂ addition to the array, signal intensities with luciferin increased as compared to the control condition for all cells transfected with pERE(3×)TK-ffLUC plasmids (See FIG. 6D, columns 3 and 4), indicating ERα-dependent transcriptional activation of the ERE-regulated plasmid. Expression of the pLUC plasmid was largely unaffected by E₂ (See FIG. 6D, column 1). Addition of the antiestrogen FUL to the arrays completely eliminated the signal in cells transfected with pERE(3×)TK-ffLUC alone (See FIG. 6F, column 3), or substantially reduced signal intensities in cells transfected with both pERE(3×)TK-ffLUC and pTK-rLUC (See FIG. 6F, column 4), in which the luminescence that was detected was again due to carryover of the VIVIREN signal. Therefore, addition of 10⁻⁶ M FUL led to a complete blockade of ERα-stimulated activity by 10⁻⁹ M E₂. pLUC expression was also lowered in the presence of FUL (See FIG. 6F, column 1) indicating that some transcriptional elements in the CMV may be indirectly regulated by ERα, possibly by ERα tethering to AP1 and SP1 proteins bound directly to DNA in this promoter.

Average renilla luminescence intensities in cells transfected with pERE(3×)TK-ffLUC and pTK-rLUC plasmids (See FIG. 6A, C, E, column 4) were similar in control and E₂+FUL treated cells, but lower in E₂ alone treated cells. Although an understanding of the mechanism is not necessary to practice the present invention and the present invention is not limited to any particular mechanism of action, in some embodiments, lower renilla luciferase activity is due to competition for transcriptional cofactors between the ERE(3×)TK and TK-only regulated promoters. For example, under E₂ stimulation conditions, ERα transcriptional coregulators and basal transcriptional machinery may be preferentially recruited to ERE-containing promoters rather than promoters lacking EREs. Hence, in cells treated with E₂, may occur at the TK-renilla luciferase promoter due to titrating out of limiting transcription factors.

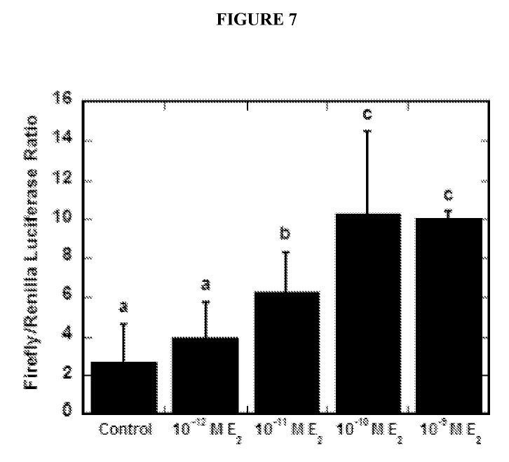

Induction of the ER-regulated plasmid system in the array mimicked results obtained through traditional assay methods. Firefly luciferase expression was determined by subtracting the VIVIREN signal from the signal obtained through imaging with D-luciferin, which was then normalized by the VIVIREN signal (See FIG. 6G). For cells transfected with both the pERE(3×)TK-ffLUC and pTK-rLUC plasmids (See FIG. 6, column 4), E₂ statistically induced dual-luciferase activity 10-fold (p<0.001) over control or FUL conditions (See FIG. 6G). Thus, the present invention provides that the arrays described herein can accurately report activity of the ERα transcription factor. The concentration response of E₂ was examined to determine the sensitivity of the reporter system in an array format (See FIG. 7). For arrays with spots containing both the pERE(3×)TK-ffLUC and pTK-rLUC plasmids, increasing the concentration of E₂ statistically increased the induction of firefly luciferase expression (p<0.05), capturing the concentration-response of E₂ in the induction of this plasmid system.

### Example 6

### Three Dimensional Transfected Cell Arrays

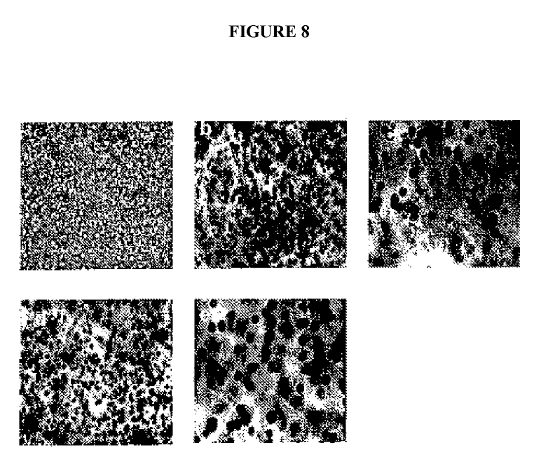

Experiments conducted during development of some embodiments of the present invention were performed to determine cell viability after encapsulation within a three-dimensional matrix, alginate. Alginate is a naturally occurring polymer produced by brown algae and has the ability to ionically crosslink in the presence of divalent cations (See, e.g., Gombotz and Wee 1998 Advanced Drug Delivery Reviews 31(3): 267-285). Cell encapsulation begins by mixing cells into a solution containing a concentration of alginate. Using a syringe, the solution is added dropwise to a 40 mM CaCl₂ and 150 mM NaCl solution. After a short period of time (e.g., 2-3 minutes) the alginate gels to form small beads, which can then be removed and placed into cell media. The present invention provides that this process is relatively mild, providing a gentle environment for cell growth and proliferation. FIG. 8 shows a phase image of MCF-7 cells encapsulated within alginate beads. The cells were stained with MTT, testing for cell viability (Live cells appear darkly stained). The present invention provides that almost all cells remain viable after 2 days of encapsulation.

A suspended solution of complexes composed of DNA plasmids and a cationic polymer was added to the alginate/cell solution prior to gelation. The cationic polymer, polyethyleimine (PEI), contains positively charged imine groups that neutralize the DNA's negatively charged phosphate backbone. Thus, in some embodiments, the neutralization allows the polymer to act as a delivery agent, facilitating transport of the DNA across the cell membrane and to the nucleus, thereby enhancing gene transfer. The present invention provides that by varying the PEI and DNA concentrations it is possible to control the nitrogen to phosphate (N:P) ratio to obtain a charge balance for optimal DNA transfection.

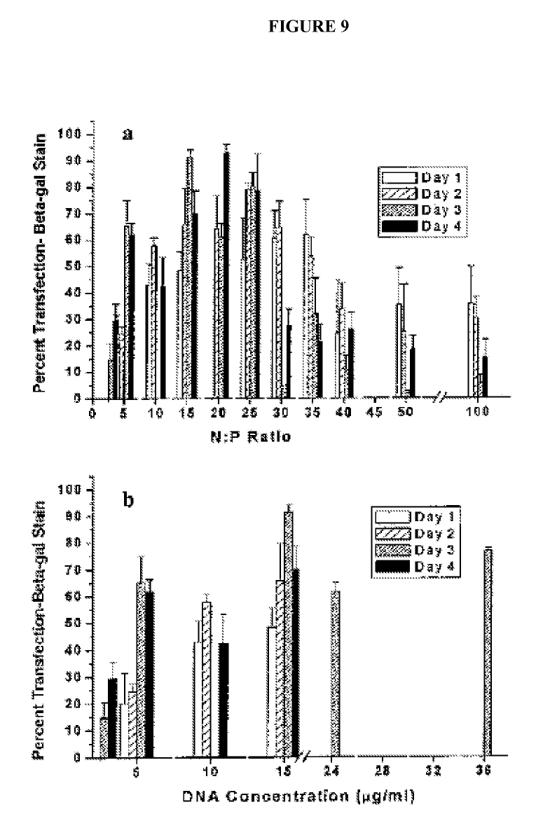

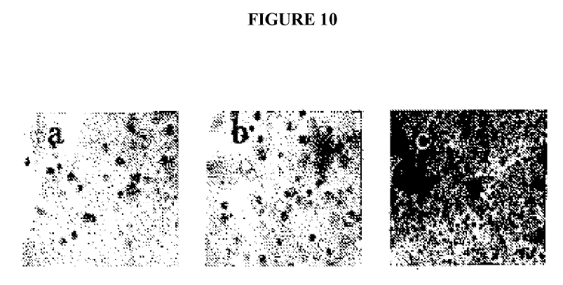

Complex suspension involved the addition of PEI to a solution containing a plasmid encoding a beta-galactosidase reporter gene. MCF-7 cells were added to the complex solution, mixed thoroughly and then this solution was added to an alginate solution. Beads were formed, placed in media and stained for beta-galactosidase transfection 1, 2, 3 and 4 days after initial incubation. This procedure was performed for 1.5% alginate beads with complexes varying in N:P ratios from 5-100, as shown in FIG. 9a. An N:P ratio of around 25 consistently gave the highest percent transfection efficiency. Using an N:P ratio of 25, transfection efficiency as a function of DNA concentration was characterized in which a maximum appeared to be reached at 12 μg/ml given a cell concentration of 250,000 cells/ml (See FIG. 2b). FIG. 10 shows phase imaging for alginate beads containing 12 μg/ml DNA in complexes with an N:P ratio of 25 and cell concentration of 250,000 cells/ml (See FIG. 10c at a lower magnification). The present invention provides, as determined by beta-galactosidase staining, a transfection efficiency of 70-90% was obtained counting only stained cells.

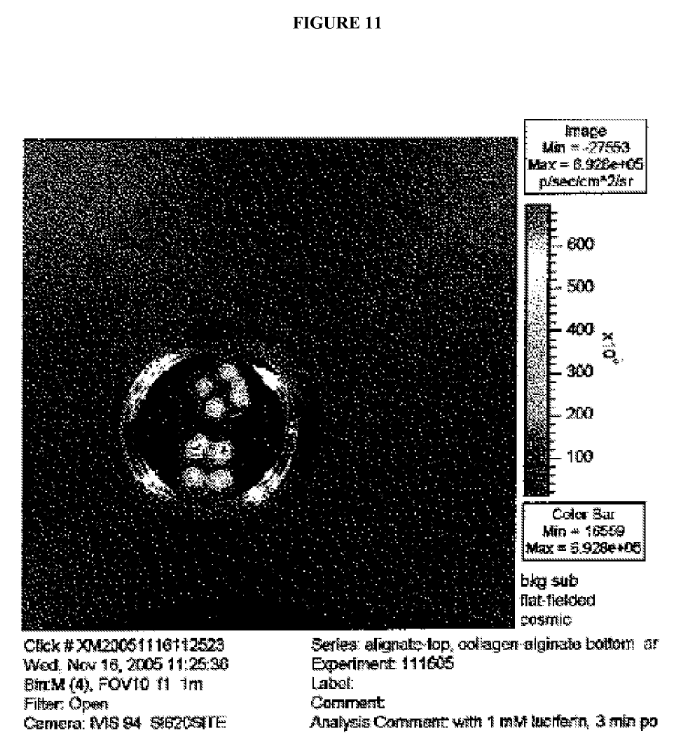

Using these optimized conditions an array of alginate and alginate/collagen beads was formed, encapsulating a mixture of MCF-7 cells and P-Luc-DNA/PEI complexes, P— Luc-DNA being plasmid containing the luciferase reporter gene. Briefly, MCF-7 cells were added to a solution of suspended complexes composed of PEI and plasmids encoding a luciferase reporter gene. The solution was mixed thoroughly and added to alginate and alginate/collagen solutions, creating mixtures with final concentrations of 1.5% alginate or 1.5% alginate with 0.2% collagen. Each mixture was then placed into a separate syringe. With a needle attached, the alginate/complex/cell mixture was added dropwise to glass coverslips and immediately submerged into a 150 mM NaCl and 40 mM CaCl₂ solution. Forming beads on a glass coverslip secured the beads to a surface while allowing them to maintain a 3D hemispherical shape. Gels were analyzed 48 hours later using real-time luciferase imaging. A 1 mM luciferin solution was added to the media above the array. Light emission was imaged 3 minutes later with an exposure time set for 1 min. FIG. 11 shows the expression for both alginate (top) and alginate/collagen (bottom) beads. Expression was confined solely to the area containing the gels

All publications and patents mentioned in the above specification are herein incorporated by reference. Various modifications and variations of the described compositions and methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

1. A composition comprising a three-dimensional transfection cell array comprising an array of spatially controllable immobilized gels, wherein said gels comprise a crosslinkable biopolymer solution comprising cells exposed to transfection molecules.

2. The composition of claim 1, wherein the gels are formed by adding a solution comprising transfection molecules and cells to an alginate solution, allowing beads to form, and placing the same within cell growth media.

3. The composition of claim 2, wherein said alginate solution comprises alginate and collagen.

4. The composition of claim 3, wherein said composition further comprises polyethyleimine (PEI).

5. The composition of claim 3, wherein the alginate is present at a final concentration of about 1.5%.

6. The composition of claim 3, wherein the collagen is present at a final concentration of about 0.2%.

7. The composition of claim 4, wherein PEI is present in an amount such that the nitrogen to phosphate ratio is 25.

8. The composition of claim 1, wherein the transfection molecules are nucleic acid sequences.

9. The composition of claim 8, wherein the nucleic acid sequences are located within expression vectors.

10. The composition of claim 1, wherein said transfection molecules comprise a normalization plasmid and a functional plasmid, wherein the normalization plasmid, comprising a promoter region and a reporter molecule, allows cellular transfection efficiency to be normalized over said array.

11. The composition of claim 10, wherein the functional plasmid comprises a reporter molecule that is different from the reporter molecule of said normalization plasmid.

12. A method of transfecting cells comprising
a) providing:
i) a composition comprising a biopolymer comprising alginate and collagen,
ii) transfection molecules, and
ii) cells; and
b) mixing the composition comprising biopolymer, transfection molecules and cells, in the presence of PEI, under conditions such that beads form; and
c) placing said beads in cell growth media.

13. A method of generating an array, comprising:
a) forming holes in a first material to generate an array mold,
b) attaching said array mold to a solid substrate to form a complex comprising a plurality of wells;
c) depositing first nucleic acid vectors into said plurality of wells under conditions such that said first nucleic acid vectors are generally immobilized on said solid substrate, wherein said first nucleic acid vectors comprise nucleic acid, and wherein said nucleic acid comprises: 1) a reporter gene; and 2) a promoter region configured to bind transcription factors; and
d) removing said array mold from said solid substrate to generate an array on said solid substrate.

14. The method of claim 13, wherein said first material comprises PDMS.

15. The method of claim 13, wherein said solid substrate comprises polystyrene.

16. The method of claim 13, prior to step d) depositing second nucleic acid vectors into said plurality of wells, wherein said second nucleic acid vectors comprise nucleic acid comprising: 1) a second reporter gene; and 2) a promoter region that allows transfection efficiency to be normalized over said array.

17. The method of claim 13, wherein said first nucleic acid vectors are immobilized on said solid substrate such that they can deliver said nucleic acid to a cell.
